(19) United States
(12) Patent Application Publication
(10) Pub. No.: US 2015/0340625 A1
(43) Pub. Date: Nov. 26, 2015

(54) ORGANIC THIN FILM TRANSISTOR, ORGANIC SEMICONDUCTOR THIN FILM, AND ORGANIC SEMICONDUCTOR MATERIAL

(71) Applicant: FUJIFILM Corporation, Tokyo (JP)

(72) Inventors: Koji TAKAKU, Kanagawa (JP); Wataru SOTOYAMA, Kanagawa (JP); Masashi KOYANAGI, Kanagawa (JP); Masaru KINOSHITA, Kanagawa (JP)

(73) Assignee: FUJIFILM CORPORATION, Tokyo (JP)

(21) Appl. No.: 14/817,842

(22) Filed: Aug. 4, 2015

Related U.S. Application Data
(63) Continuation of application No. PCT/JP2014/052868, filed on Feb. 7, 2014.

(30) Foreign Application Priority Data
Feb. 7, 2013 (JP) 2013-022484
Feb. 5, 2014 (JP) 2014-020141

(51) Int. Cl. H01L 51/00; C07D 491/048; C07D 495/04

(52) U.S. Cl. CPC H01L 51/0071; C07D 495/04; C07D 491/048; H01L 51/0068; H01L 51/0558

(57) ABSTRACT

An organic thin film transistor containing a compound represented by one of the following formulae in a semiconductor active layer has a high carrier mobility and a small change in the threshold voltage after repeated driving. X represents S or O, Z represents a substituent having a length of 3.7 Å or less, and at least one of R¹ to R⁸ represents -L-R wherein L represents alkylene, etc., and R represents alkyl, etc.

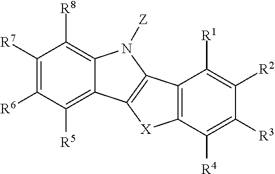

### CROSS-REFERENCE TO RELATED APPLICATIONS

This application is a Continuation of PCT International Application No. PCT/JP2014/052868 filed on Feb. 7, 2014, which claims priority under 35 U.S.C. Section 119(a) to Japanese Patent Application No. 2013-022484 filed on Feb. 7, 2013, and Japanese Patent Application No. 2014-020141 filed on Feb. 5, 2014. Each of the above applications is hereby expressly incorporated by reference, in its entirety, into the present application.

### BACKGROUND OF THE INVENTION

1. Field of the Invention

The present invention relates to an organic thin film transistor, an organic semiconductor thin film, and an organic semiconductor material. More specifically, the invention relates to a compound having a benzothienoindole or benzofuranoindole structure, an organic thin film transistor containing the compound, an organic semiconductor material for a non-light emitting organic semiconductor device containing the compound, a material for an organic thin film transistor containing the compound, a coating solution for a non-light emitting organic semiconductor device containing the compound, and an organic semiconductor thin film for a non-light emitting organic semiconductor device containing the compound.

2. Background Art

A device using an organic semiconductor material is expected to have various advantages as compared to an ordinary device using an inorganic semiconductor material, such as silicon, and thus is receiving much attention. Examples of the device using an organic semiconductor material include a photoelectric conversion device, such as an organic thin film solar cell and a solid state image sensing device, using an organic semiconductor material as a photoelectric conversion material, and a non-light emitting organic transistor. The device using an organic semiconductor material has a possibility that a device having a large area may be produced at a low temperature and a low cost, as compared to a device using an inorganic semiconductor material. Furthermore, the characteristics of the material may be easily changed by changing the molecular structure thereof, and thus there is a wide range of variations in materials, by which functions and devices that have not been achieved by an inorganic semiconductor material may be realized.

In the devices, an organic thin film transistor, an organic semiconductor thin film and an organic semiconductor material are particularly demanded. Patent Documents 1 and 2 describe the use of a heteroacene dimer having a benzothienoindole or benzofuranoindole structure in an organic transistor, and describe that an organic transistor that has a high carrier mobility and a large current on/off ratio and is excellent in storage stability may be provided.

A compound having a benzothienoindole or benzofuranoindole structure has also been used in an organic electroluminescent device, an organic photoelectric conversion device, an organic thin film solar cell, and the like. For example, Patent Document 3 describes a compound having a benzothienoindole or benzofuranoindole structure and having as a substituent on the N atom a butyl group, an aryl group, a heteroaryl group or the like, and describes that the use thereof as a host material of an organic electroluminescent (which may also be referred to as organic EL) device may improve the light emission efficiency of the device, thereby providing an organic EL device that sufficiently ensures the driving stability. However, Patent Document 3 does not describe the use of the compound having such a structure in an organic thin film transistor.

Patent Document 4 describes a compound having a benzothienoindole or benzofuranoindole structure and having as a substituent on the N atom a hexyl group, and describes that an organic photoelectric conversion device, a solar cell and a photosensor array that have a high conversion efficiency and high durability may be provided.

Patent Document 5 describes a benzothienoindole derivative having as a substituent on the N atom a butyl group, a hexyl group, a phenyl group, an anthracenyl group, a pyrenyl group, a thienyl group or the like, and describes a compound that is stable to light, oxygen and heat, and shows photoelectric conversion characteristics with a high efficiency on using in an organic thin film solar cell. Patent Document 5 describes in the example thereof investigations of various characteristics only on a benzothienoindole derivative that has as a substituent on the N atom a phenyl group or an anthracenyl group.

However, Patent Documents 4 and 5 do not describe the use of the compounds having such structures in an organic thin film transistor.

Non-patent Document 1 describes a compound having a benzothienoindole or benzofuranoindole structure and having a methyl group as a substituent on the N atom, and a —CO₂C₂H₅ group on the side chain thereof. Non-patent Document 1 describes that the compound may be used as a photoconductor material represented by an organic photosensitive material.

### CITATION LIST

### Patent Documents

Patent Document 1: JP-A-2009-182034
Patent Document 2: JP-A-2010-177644
Patent Document 3: WO 2012/035934
Patent Document 4: WO 2010/041687
Patent Document 5: JP-A-2010-270084

### Non-Patent Document

Non-patent Document 1: Journal of Organic Chemistry, 58 (19), 5209-20 (1993)

### SUMMARY OF INVENTION

It has been known that a polycyclic condensed compound containing an aromatic heterocyclic ring is useful as a material for an organic EL device, as described in Patent Document 3. However, it may not be said that a compound that is useful as a material for an organic EL device is immediately useful as a semiconductor material for an organic thin film transistor. This is because there is a difference in the characteristics demanded for the organic compound between an organic EL device and an organic thin film transistor. An organic EL device generally requires charge transport in the thickness direction of the thin film (which is generally from several nanometers to several hundred nanometers), whereas an organic thin film transistor requires charge (carrier) transport in a long distance between electrodes in the plane direction of the thin film (which is generally from several micrometers to several hundred micrometers). Accordingly, the demanded carrier mobility is considerably high. Thus, as a semiconductor material for an organic thin film transistor, an organic compound that has a high alignment order of molecules with high crystallinity is demanded. Furthermore, for achieving a high carrier mobility, the π-conjugate plane is preferably perpendicular to the substrate. In an organic EL device, on the other hand, a device that has a high light emission efficiency and uniform in-plane light emission is demanded for enhancing the light emission efficiency. In general, an organic compound having high crystallinity may be a cause of light emission defects, such as in-plane electric field unevenness, in-plane light emission unevenness and light emission quenching, and thus the material for an organic EL device is demanded to have high amorphous property with low crystallinity. Accordingly, even when an organic compound constituting a material for an organic EL device is diverted to an organic semiconductor material, good transistor characteristics may not immediately obtained.

The heteroacene dimer described in Patent Document 1 is difficult to form a herringbone structure showing a high mobility and may not provide a high carrier mobility. The examples of Patent Documents 1 and 2 describe relatively high mobilities, but according to the investigations made by the present inventors, it has been found that the compounds described therein used in a BC device (which is an abbreviation of a bottom-contact device) show only such a low mobility as approximately 1×10⁻³ cm/V·s. Furthermore, in the case where an organic thin film transistor using the heteroacene dimer described in Patent Document 2 is subjected to repeated driving, the investigations made by the inventors reveal that the change in the threshold voltage becomes large in the repeated driving. The large change in the threshold voltage brings about a problem that the transistor is deteriorated in reliability and may not be used for a prolonged period of time. The change in the threshold voltage after repeated driving is a problem that has not been known in the art.

The benzothienoindole or benzofuranoindole compound having a substituent, such as a butyl group or a hexyl group, on the N atom described in Patent Documents 3 and 4 undergoes an increased intermolecular distance due to the bulky substituent on the N atom to fail to provide a sufficient overlap of HOMO, and thus a high carrier mobility may not be obtained. Furthermore, Patent Documents 3 and 4 do not contain examples of applying the compound to an organic transistor, and the investigations made by the inventors reveal that an organic thin film transistor that is produced by using the compound described in Patent Document 3 has a low carrier mobility and shows a large change in the threshold voltage after repeated driving, and that an organic thin film transistor that is produced by using the compound described in Patent Document 4 has a low carrier mobility, both of which fail to provide sufficient transistor characteristics.

The benzothienoindole compound having a bulky aryl group as a substituent on the N atom described in Patent Document 5 undergoes an increased intermolecular distance due to the bulky substituent on the N atom to fail to provide a sufficient overlap of HOMO, and thus a high carrier mobility may not be obtained. Furthermore, Patent Document 5 does not contain an example of applying the compound to an organic transistor, and the investigations made by the inventors reveal that an organic thin film transistor that is produced by using the compound described in Patent Document 5 has a low carrier mobility to fail to provide sufficient transistor characteristics.

Under the circumstances, the inventors have made investigations for solving the problems in the related art. An object to be achieved by the invention is to provide an organic thin film transistor that has a high carrier mobility and a small change in the threshold voltage after repeated driving.

As a result of earnest investigations for solving the problems, the inventors have found that an organic thin film that has high crystallinity and is advantageous for carrier transport may be obtained by introducing a substituent promoting a herringbone molecular orientation into a compound having a benzothienoindole or benzofuranoindole structure. The inventors have found that the compound having the N atom that is unsubstituted or has a non-bulky substituent undergoes an intermolecular distance that is not excessively increased to provide a sufficient overlap of HOMO, and thus a high carrier mobility may be obtained.

Furthermore, the inventors have found that an organic thin film transistor that uses the benzothienoindole or benzofuranoindole derivative having the substituent having the particular structure in a semiconductor active layer shows a small change in the threshold voltage after repeated driving, and thus have completed the invention.

The invention as a specific measure for solving the problems includes the following aspects.

(1) An organic thin film transistor containing a compound represented by the following formula (1) in a semiconductor active layer:

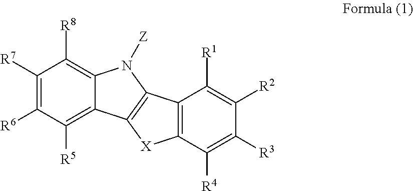

wherein in the formula (1), X represents a S atom or an O atom; Z represents a substituent that has a length of 3.7 Å or less from the N atom to the end of the substituent; and R¹ to R⁸ each independently represent a hydrogen atom or a substituent, provided that at least one of R¹ to R⁸ represents a substituent represented by the following formula (W):

-L-R Formula (W)

wherein in the formula (W), L represents a divalent linking group represented by any one of the following formulae (L-1) to (L-12), or a divalent linking group containing 2 or more divalent linking groups each represented by any one of the following formulae (L-1) to (L-12) bonded to each other; and R represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R has a number of carbon atoms of 2 or more in the case where L represents a divalent linking group represented by any one of the formulae (L-1) to (L-3), or 4 or more in the case where L represents a divalent linking group represented by any one of the formulae (L-4) to (L-12), and R represents a substituted or unsubstituted trialkylsilyl group only in the case where L adjacent to R represents a divalent linking group represented by the formula (L-3):

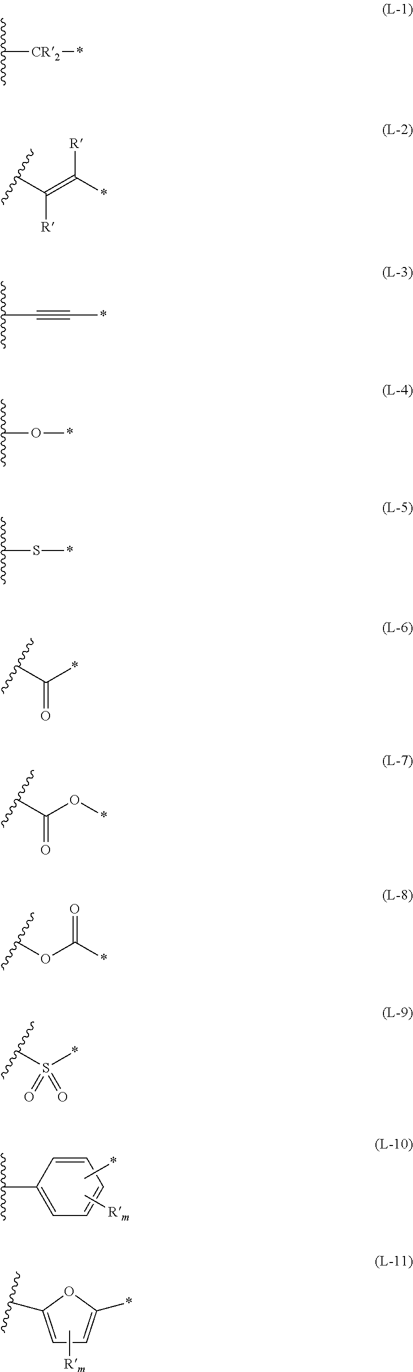

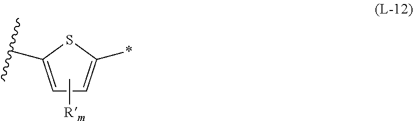

wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

(2) In the organic thin film transistor according to the item (1), at least one of R², R³, R⁶ and R⁷ preferably represents a substituent represented by the formula (W).

(3) In the organic thin film transistor according to the item (1), the compound represented by the formula (1) is preferably a compound represented by the following formula (2-1) or (2-2):

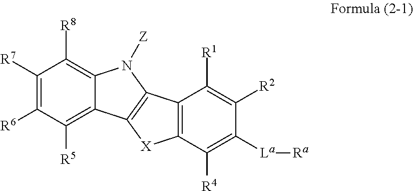

wherein in the formula (2-1), X represents a S atom or an O atom; Z represents a substituent that has a length of 3.7 Å or less from the N atom to the end of the substituent; R¹, R² and R⁴ to R⁸ each independently represent a hydrogen atom or a substituent, provided that R⁷ is not a substituent represented by -L^{a}-R^{a}; L^{a} represents a divalent linking group represented by any one of the following formulae (L-1) to (L-12), or a divalent linking group containing 2 or more divalent linking groups each represented by any one of the following formulae (L-1) to (L-12) bonded to each other; and R^{a} represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R^{a} has a number of carbon atoms of 2 or more in the case where L^{a} represents a divalent linking group represented by any one of the formulae (L-1) to (L-3), or 4 or more in the case where L^{a} represents a divalent linking group represented by any one of the formulae (L-4) to (L-12), and R^{a} represents a substituted or unsubstituted trialkylsilyl group only in the case where L^{a} adjacent to R^{a} represents a divalent linking group represented by the formula (L-3),

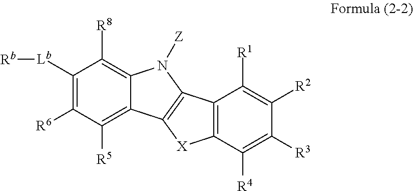

wherein in the formula (2-2), X represents a S atom or an O atom; Z represents a substituent that has a length of 3.7 Å or less from the N atom to the end of the substituent; R¹ to R⁶ and R⁸ each independently represent a hydrogen atom or a substituent, provided that R³ is not a substituent represented by -L^{b}-R^{b}; L^{b} represents a divalent linking group represented by any one of the following formulae (L-1) to (L-12), or a divalent linking group containing 2 or more divalent linking groups each represented by any one of the following formulae (L-1) to (L-12) bonded to each other; and R^{b} represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R^{b} has a number of carbon atoms of 2 or more in the case where L^{b} represents a divalent linking group represented by any one of the formulae (L-1) to (L-3), or 4 or more in the case where L^{b} represents a divalent linking group represented by any one of the formulae (L-4) to (L-12), and R^{b} represents a substituted or unsubstituted trialkylsilyl group only in the case where L^{b} adjacent to R^{b} represents a divalent linking group represented by the formula (L-3):

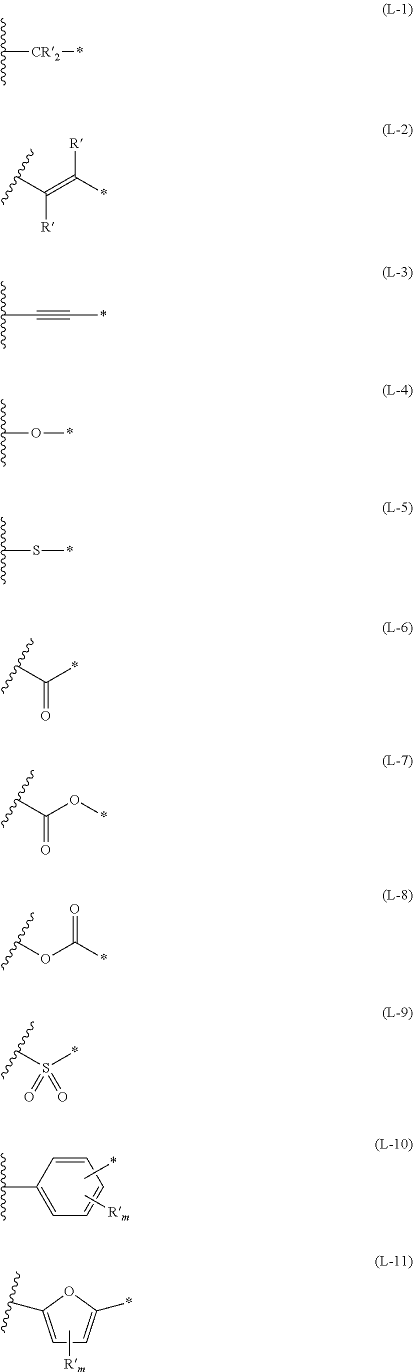

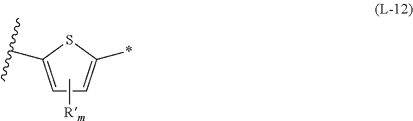

wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

(4) In the organic thin film transistor according to the item (1), the compound represented by the formula (1) is preferably a compound represented by the following formula (3):

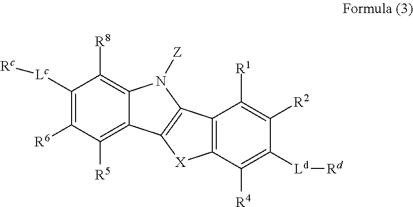

wherein in the formula (3), X represents a S atom or an O atom; Z represents a substituent that has a length of 3.7 Å or less from the N atom to the end of the substituent; R¹, R², R⁴ to R⁶ and R⁸ each independently represent a hydrogen atom or a substituent; L^{c} and L^{d} each independently represent a divalent linking group represented by any one of the following formulae (L-1) to (L-12), or a divalent linking group containing 2 or more divalent linking groups each represented by any one of the following formulae (L-1) to (L-12) bonded to each other; and R^{c} and R^{d} each independently represent a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl groups represented by R^{c} and R^{d} each have a number of carbon atoms of 2 or more in the case where L^{c} and L^{d} respectively represent a divalent linking group represented by any one of the formulae (L-1) to (L-3), or 4 or more in the case where L^{c} and L^{d} respectively represent a divalent linking group represented by any one of the formulae (L-4) to (L-12), and R^{c} and R^{d} each represent a substituted or unsubstituted trialkylsilyl group only in the case where L^{c} and L^{d} adjacent to R^{c} and R^{d} respectively represent a divalent linking group represented by the formula (L-3)

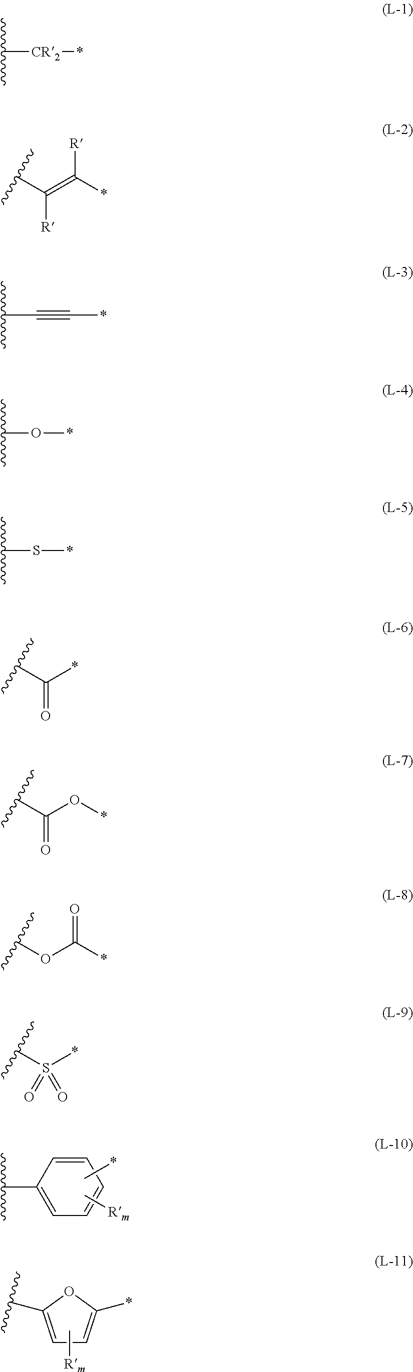

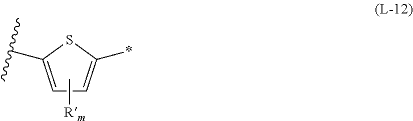

wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

(5) In the organic thin film transistor according to the item (3) or (4), in the formula (2-1), (2-2) or (3), Z preferably represents a hydrogen atom, a substituted or unsubstituted alkyl group having 2 or less carbon atoms, a substituted or unsubstituted alkynyl group having 2 or less carbon atoms, a substituted or unsubstituted alkenyl group having 2 or less carbon atoms, or a substituted or unsubstituted acyl group having 2 or less carbon atoms.

(6) In the organic thin film transistor according to any one of the items (3) to (5), in the formula (2-1), (2-2) or (3), R¹, R⁴, R⁵ and R⁸ each preferably independently represent a hydrogen atom, a fluorine atom, a substituted or unsubstituted alkyl group having from 1 to 3 carbon atoms, a substituted or unsubstituted alkynyl group having from 2 to 3 carbon atoms, a substituted or unsubstituted alkenyl group having from 2 to 3 carbon atoms, a substituted or unsubstituted alkoxy group having from 1 to 2 carbon atoms, or a substituted or unsubstituted methylthio group.

(7) In the organic thin film transistor according to any one of the items (3) to (6), in the formula (2-1), (2-2) or (3), all of L^{a}, L^{b}, L^{c} and L^{d} each preferably represent a divalent linking group represented by any one of the formulae (L-1) to (L-3), (L-10), (L-11) or (L-12), or a divalent linking group containing 2 or more of the divalent linking groups bonded to each other.

(8) In the organic thin film transistor according to any one of the items (3) to (7), in the formula (2-1), (2-2) or (3), all of L^{a}, L^{b}, L^{c} and L^{d} each preferably independently represent a divalent linking group represented by the formula (L-1) or (L-10).

(9) In the organic thin film transistor according to any one of the items (3) to (8), in the formula (2-1), (2-2) or (3), all R^{a}, R^{b}, R^{c} and R^{d} each preferably independently represent a substituted or unsubstituted alkyl group.

(10) In the organic thin film transistor according to the item (3), it is preferred that in the formula (2-1) or (2-2), R^{a} and R^{b} each independently represent a branched alkyl group; or

L^{a} and L^{b} each independently represent a divalent linking group represented by the formula (L-1), and at least one of R′ in the divalent linking groups represented by the formula (L-1) represents an alkyl group.

(11) In the organic thin film transistor according to the item (4), it is preferred that in the formula (3), R^{c} and R^{d} each independently represent a linear alkyl group, provided that in the case where L^{c} and L^{d} each independently represent a divalent linking group represented by the formula (L-1), all R′ in the divalent linking groups represented by the formula (L−1) each represent a hydrogen atom.

(12) A compound represented by the following formula (1):

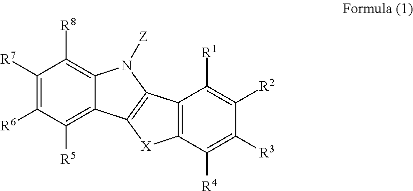

wherein in the formula (1), X represents a S atom or an O atom; Z represents a substituent that has a length of 3.7 Å or less from the N atom to the end of the substituent; and R¹ to R⁸ each independently represent a hydrogen atom or a substituent, provided that at least one of R¹ to R⁸ represents a substituent represented by the following formula (W):

-L-R Formula (W)

wherein in the formula (W), L represents a divalent linking group represented by any one of the following formulae (L-1) to (L-12), or a divalent linking group containing 2 or more divalent linking groups each represented by any one of the following formulae (L-1) to (L-12) bonded to each other; and R represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R has a number of carbon atoms of 2 or more in the case where L represents a divalent linking group represented by any one of the formulae (L-1) to (L-3), or 4 or more in the case where L represents a divalent linking group represented by any one of the formulae (L-4) to (L-12), and R represents a substituted or unsubstituted trialkylsilyl group only in the case where L adjacent to R represents a divalent linking group represented by the formula (L-3):

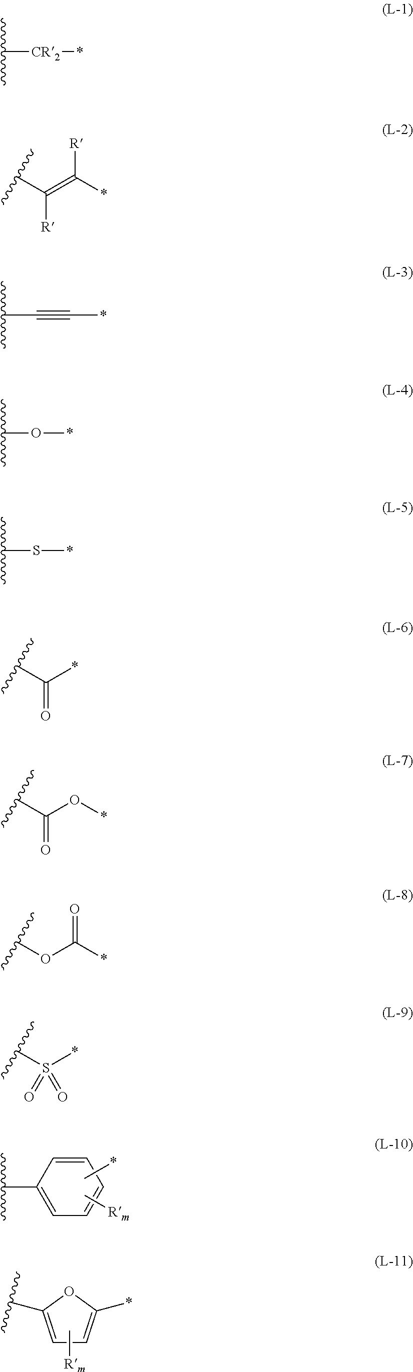

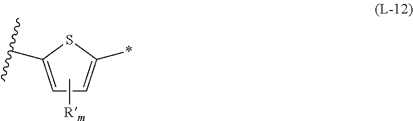

wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12) R′ each independently represent a hydrogen atom or a substituent.

(13) In the compound according to the item (12), at least one of R², R³, R⁶ and R⁷ preferably represents a substituent represented by the formula (W).

(14) In the compound according to the item (1), the compound represented by the formula (1) is preferably a compound represented by the following formula (2-1) or (2-2)

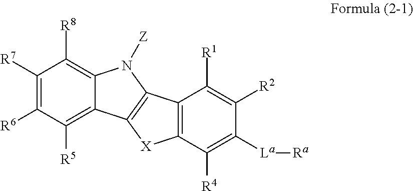

wherein in the formula (2-1), X represents a S atom or an O atom; Z represents a substituent that has a length of 3.7 Å or less from the N atom to the end of the substituent; R¹, R² and R⁴ to R⁸ each independently represent a hydrogen atom or a substituent, provided that R⁷ is not a substituent represented by -L^{a}-R^{a}; L^{a} represents a divalent linking group represented by any one of the following formulae (L-1) to (L-12), or a divalent linking group containing 2 or more divalent linking groups each represented by any one of the following formulae (L-1) to (L-12) bonded to each other; and R^{a} represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R^{a} has a number of carbon atoms of 2 or more in the case where L^{a} represents a divalent linking group represented by any one of the formulae (L-1) to (L-3), or 4 or more in the case where L^{a} represents a divalent linking group represented by any one of the formulae (L-4) to (L-12), and R^{a} represents a substituted or unsubstituted trialkylsilyl group only in the case where L^{a} adjacent to R^{a} represents a divalent linking group represented by the formula (L-3),

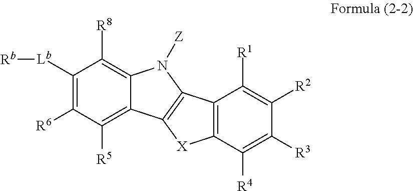

wherein in the formula (2-2), X represents a S atom or an O atom; Z represents a substituent that has a length of 3.7 Å or less from the N atom to the end of the substituent; R¹ to R⁶ and R⁸ each independently represent a hydrogen atom or a substituent, provided that R³ is not a substituent represented by -L^{b}-R^{b}; L^{b} represents a divalent linking group represented by any one of the following formulae (L-1) to (L-12), or a divalent linking group containing 2 or more divalent linking groups each represented by any one of the following formulae (L-1) to (L-12) bonded to each other; and R^{b} represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R^{b} has a number of carbon atoms of 2 or more in the case where L^{b} represents a divalent linking group represented by any one of the formulae (L-1) to (L-3), or 4 or more in the case where L^{b} represents a divalent linking group represented by any one of the formulae (L-4) to (L-12), and R^{b} represents a substituted or unsubstituted trialkylsilyl group only in the case where L^{b} adjacent to R^{b} represents a divalent linking group represented by the formula (L-3):

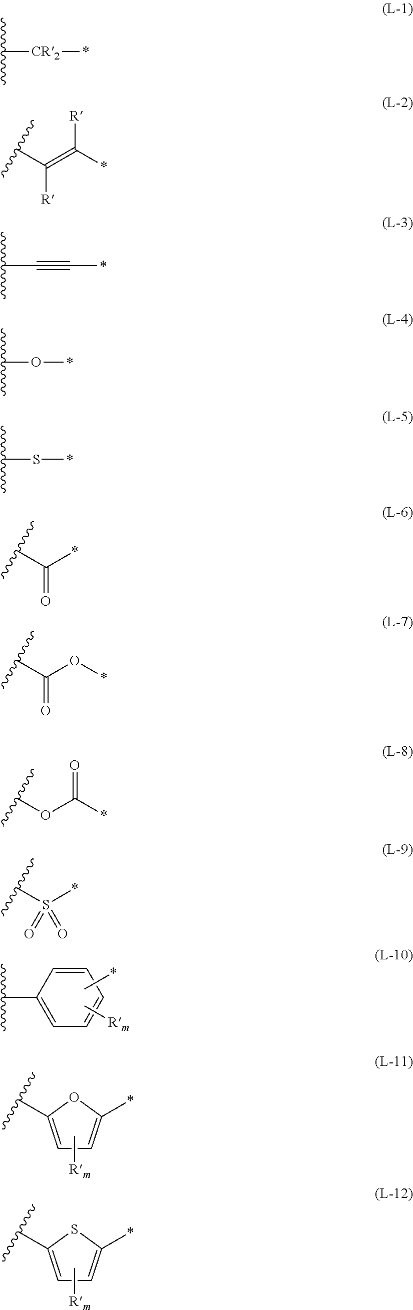

wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

(15) In the compound according to the item (12), the compound represented by the formula (1) is preferably a compound represented by the following formula (3):

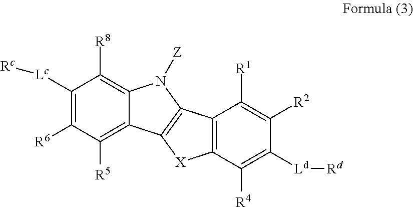

wherein in the formula (3), X represents a S atom or an O atom; Z represents a substituent that has a length of 3.7 Å or less from the N atom to the end of the substituent; R¹, R², R⁴ to R⁶ and R⁸ each independently represent a hydrogen atom or a substituent; L^{c} and L^{d} each independently represent a divalent linking group represented by any one of the following formulae (L-1) to (L-12), or a divalent linking group containing 2 or more divalent linking groups each represented by any one of the following formulae (L-1) to (L-12) bonded to each other; and R^{c} and R^{d} each independently represent a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl groups represented by R^{c} and R^{d} each have a number of carbon atoms of 2 or more in the case where L^{c} and L^{d} respectively represent a divalent linking group represented by any one of the formulae (L-1) to (L-3), or 4 or more in the case where L^{c} and L^{d} respectively represent a divalent linking group represented by any one of the formulae (L-4) to (L-12), and R^{c} and R^{d} each represent a substituted or unsubstituted trialkylsilyl group only in the case where L^{c} and L^{d} adjacent to R^{c} and R^{d} respectively represent a divalent linking group represented by the formula (L-3):

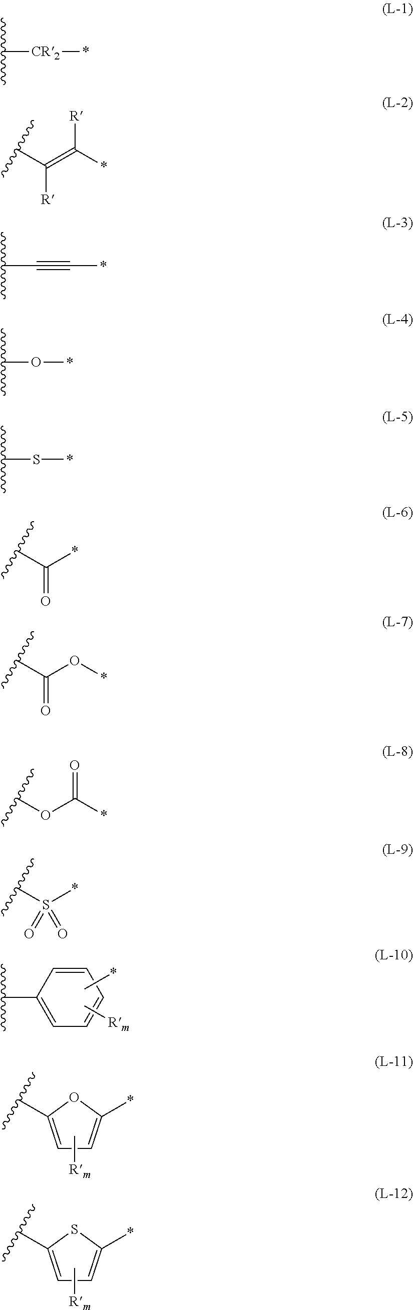

wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

(16) In the compound according to the item (14) or (15), in the formula (2-1), (2-2) or (3), Z preferably represents a hydrogen atom, a substituted or unsubstituted alkyl group having 2 or less carbon atoms, a substituted or unsubstituted alkynyl group having 2 or less carbon atoms, a substituted or unsubstituted alkenyl group having 2 or less carbon atoms, or a substituted or unsubstituted acyl group having 2 or less carbon atoms.

(17) In the compound according to any one of the items (14) to (16), in the formula (2-1), (2-2) or (3), R¹, R⁴, R⁵ and R⁸ each preferably independently represent a hydrogen atom, a fluorine atom, a substituted or unsubstituted alkyl group having from 1 to 3 carbon atoms, a substituted or unsubstituted alkynyl group having from 2 to 3 carbon atoms, a substituted or unsubstituted alkenyl group having from 2 to 3 carbon atoms, a substituted or unsubstituted alkoxy group having from 1 to 2 carbon atoms, or a substituted or unsubstituted methylthio group.

(18) In the compound according to any one of the items (14) to (17), in the formula (2-1), (2-2) or (3), all of L^{a}, L^{b}, L^{c} and L^{d} each preferably represent a divalent linking group represented by any one of the formulae (L-1) to (L-3), (L-10), (L-11) or (L-12), or a divalent linking group containing 2 or more of the divalent linking groups bonded to each other.

(19) In the compound according to any one of the items (14) to (18), in the formula (2-1), (2-2) or (3), all of L^{a}, L^{b}, L^{e} and L^{d} each preferably independently represent a divalent linking group represented by the formula (L-1) or (L-10).

(20) In the compound according to any one of the items (14) to (19), in the formula (2-1), (2-2) or (3), all R^{a}, R^{b}, R^{c} and R^{d} each preferably independently represent a substituted or unsubstituted alkyl group.

(21) In the compound according to the item (14), it is preferred that in the formula (2-1) or (2-2), R^{a} and R^{b} each independently represent a branched alkyl group; or

L^{a} and L^{b} each independently represent a divalent linking group represented by the formula (L-1), and at least one of R′ in the divalent linking groups represented by the formula (L-1) represents an alkyl group.

(22) In the compound according to the item (15), it is preferred that in the formula (3), R^{c} and R^{d} each independently represent a linear alkyl group, provided that in the case where L^{c} and L^{d} each independently represent a divalent linking group represented by the formula (L-1), all R′ in the divalent linking groups represented by the formula (L-1) each represent a hydrogen atom.

(23) An organic semiconductor material for a non-light emitting organic semiconductor device, containing the compound represented by the formula (1) according to any one of the items (12) to (22).

(24) A material for an organic thin film transistor, containing the compound represented by the formula (1) according to any one of the items (12) to (22).

(25) A coating solution for a non-light emitting organic semiconductor device, containing the compound represented by the formula (1) according to any one of the items (12) to (22).

(26) A coating solution for a non-light emitting organic semiconductor device, containing the compound represented by the formula (1) according to anyone of the items (12) to (22), and a polymer binder.

(27) An organic semiconductor thin film for a non-light emitting organic semiconductor device, containing the compound represented by the formula (1) according to anyone of the items (12) to (22).

(28) An organic semiconductor thin film for a non-light emitting organic semiconductor device, containing the compound represented by the formula (1) according to anyone of the items (12) to (22), and a polymer binder.

(29) The organic semiconductor thin film for a non-light emitting organic semiconductor device according to the item (27) or (28) is preferably produced by a solution coating method.

According to the invention, an organic thin film transistor may be provided that has a high carrier mobility and a small change in the threshold voltage after repeated driving.

### BRIEF DESCRIPTION OF DRAWINGS

FIG. 1 is a schematic illustration showing a cross sectional structure of one example of the organic thin film transistor of the invention.

FIG. 2 is a schematic illustration showing a cross sectional structure of an organic thin film transistor that is produced as a substrate for measuring FET characteristics in the example of the invention.

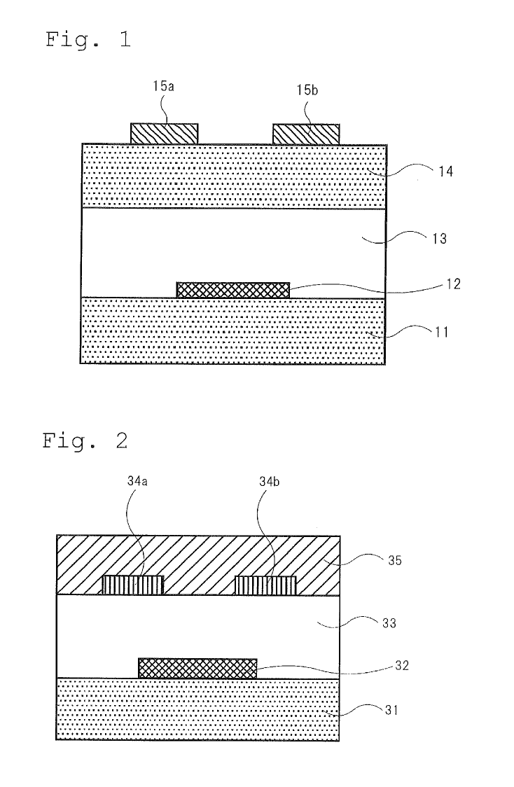

In FIGS. 1 and 2, 11 is substrate, 12 is electrode, 13 is insulating layer, 14 is semiconductor active layer (organic material layer or organic semiconductor layer), 15a, 15b are each electrode, 31 is substrate, 32 is electrode, 33 is insulating layer, 34a, 34b are each electrode, and 35 is semiconductor active layer (organic material layer or organic semiconductor layer).

### DESCRIPTION OF EMBODIMENTS

The invention will be described in detail below. The description for the constitutional components shown below may be made with reference to representative embodiments and specific examples, but the invention is not limited to the embodiments and the examples. In the description, a numerical range expressed with reference to an upper limit and/or a lower limit means a range that includes the upper limit and/or the lower limit.

In the invention, the hydrogen atom that is referred without any particular discrimination in the description of the formulae herein includes isotopes thereof (such as a deuterium atom). The atoms constituting the substituents also include isotopes thereof.

### Organic Thin Film Transistor

The organic thin film transistor of the invention contains a compound represented by the following formula (1) in a semiconductor active layer:

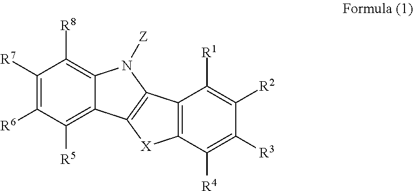

wherein in the formula (1), X represents a S atom or an O atom; Z represents a substituent that has a length of 3.7 Å or less from the N atom to the end of the substituent; and R¹ to R⁸ each independently represent a hydrogen atom or a substituent, provided that at least one of R¹ to R⁸ represents a substituent represented by the following formula (W):

-L-R Formula (W)

wherein in the formula (W), L represents a divalent linking group represented by any one of the following formulae (L-1) to (L-12), or a divalent linking group containing 2 or more divalent linking groups each represented by any one of the following formulae (L-1) to (L-12) bonded to each other; and R represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R has a number of carbon atoms of 2 or more in the case where L represents a divalent linking group represented by any one of the formulae (L-1) to (L-3), or 4 or more in the case where L represents a divalent linking group represented by any one of the formulae (L-4) to (L-12), and R represents a substituted or unsubstituted trialkylsilyl group only in the case where L adjacent to R represents a divalent linking group represented by the formula (L-3):

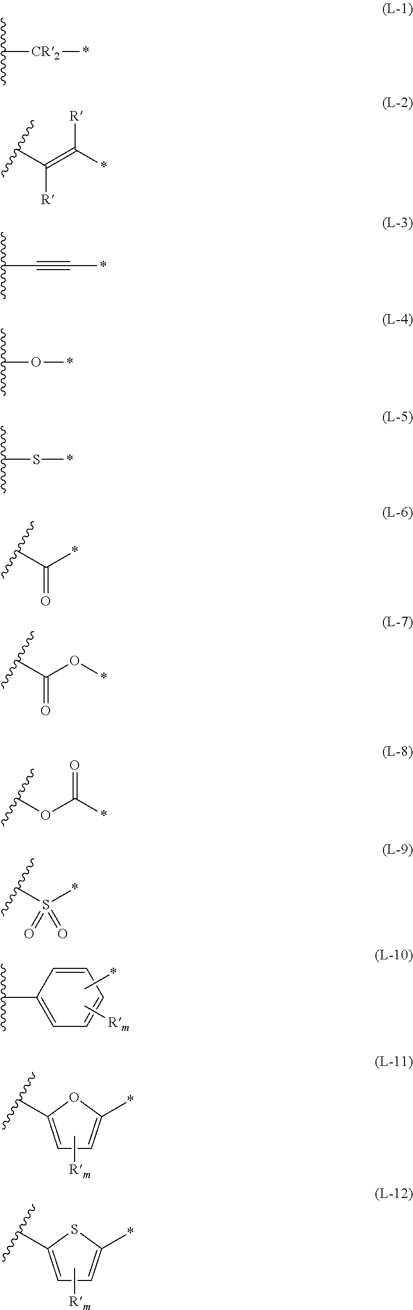

wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

According to the constitution, the organic thin film transistor of the invention has a high carrier mobility and a small change in the threshold voltage after repeated driving.

The compound represented by the formula (1) has a substituent represented by the formula (W) as at least one of R¹ to R⁸, and thus is preferred from the standpoint of the applicability of the material to a solution process and the molecular orientation in the film, and a semiconductor material capable of forming an organic thin film that has high crystallinity and is advantageous for carrier transport. According to the structure, an organic thin film transistor having a high carrier mobility may be obtained. Moreover, the production efficiency of the organic thin film capable of being applied to an organic thin film transistor may be enhanced to suppress the production cost thereof, and the chemical and physical stability of the thin film may also be enhanced.

For reducing the change in the threshold voltage after repeated driving, there are such requirements as chemical stability of the organic semiconductor material (particularly, air oxidation resistance and redox stability), thermal stability in the form of a thin film, a large film density capable of preventing air and water from invading, a film quality with less defects capable of preventing charges from being accumulated, and the like. It is considered that the compound represented by the formula (1) satisfies these requirements and thus has a small change in the threshold voltage after repeated driving. Accordingly, the organic thin film transistor of the invention having a less change in the threshold voltage after repeated driving has a semiconductor active layer that has a high chemical stability, a high film density, and the like, and thus effectively functions as a transistor for a prolonged period of time.

It is considered that the organic semiconductor material using the compound represented by the formula (1) forms a herringbone structure suitable for carrier transport in the organic thin film, and is liable to form a two-dimensional overlap of the orbitals (the advantage of a herringbone structure for carrier transport is described, for example, in Adv. Mater., 2011, 23, 4347-4370, and the like). It is considered that according to the structure, the compound of the invention may achieve good film quality and a high carrier mobility, and thus may be preferably used in an organic thin film transistor.

Preferred embodiments of the compound of the invention, the organic thin film transistor of the invention and the like will be described below.

### Compound Represented by Formula (1)

The compound of the invention is represented by the following formula (1). The compound of the invention is contained in a semiconductor active layer described later in the organic thin film transistor of the invention. Thus, the compound of the invention may be used as a material for an organic thin film transistor.

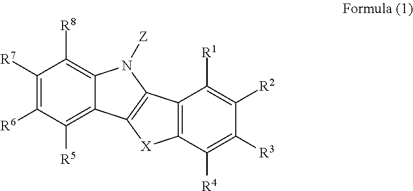

wherein in the formula (1), X represents a S atom or an O atom; Z represents a substituent that has a length of 3.7 Å or less from the N atom to the end of the substituent; and R¹ to R⁸ each independently represent a hydrogen atom or a substituent, provided that at least one of R¹ to R⁸ represents a substituent represented by the following formula (W):

-L-R Formula (W)

wherein in the formula (W), L represents a divalent linking group represented by any one of the following formulae (L-1) to (L-12), or a divalent linking group containing 2 or more divalent linking groups each represented by any one of the following formulae (L-1) to (L-12) bonded to each other; and R represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R has a number of carbon atoms of 2 or more in the case where L represents a divalent linking group represented by any one of the formulae (L-1) to (L-3), or 4 or more in the case where L represents a divalent linking group represented by any one of the formulae (L-4) to (L-12), and R represents a substituted or unsubstituted trialkylsilyl group only in the case where L adjacent to R represents a divalent linking group represented by the formula (L-3):

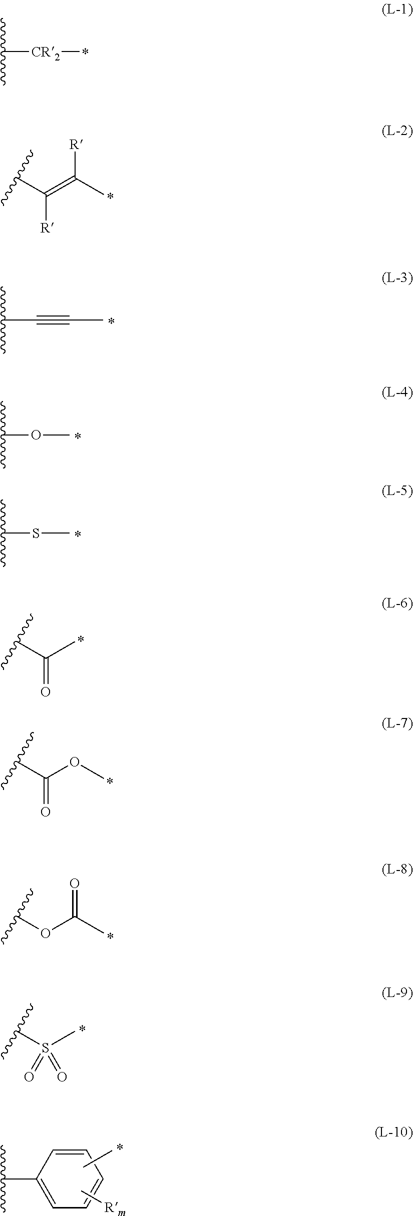

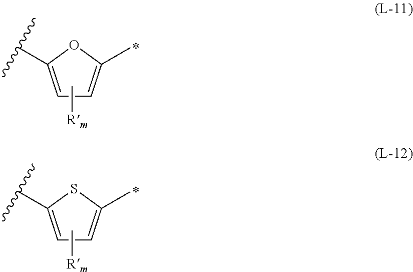

wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

In the formula (1), Z represents a substituent that has a length of 3.7 Å or less from the N atom to the end of the substituent. The molecular length of the substituent Z herein means the length of from the N atom in the N—Z bond in the benzothienoindole or benzofuranoindole structure to the end of the substituent represented by Z. The structure optimization calculation may be performed by the density functional approach (Gaussian 03 (Gaussian, Inc., U.S.), base function: 6-31G*, exchange correlation function: B3LYP/LANL2DZ). In the formula (1), Z preferably represents a substituent that has a length of from 1.0 to 3.7 Å from the N atom to the end of the substituent, and more preferably a substituent that has a length of from 1.0 to 2.2 Å. The molecular lengths of representative substituents are 4.6 Å for a propyl group, 4.6 Å for a pyrrol group, 4.5 Å for a propynyl group, 4.6 Å for propenyl group, 4.5 Å for an ethoxy group, 3.7 Å for a methylthio group, 3.4 Å for an ethenyl group, 3.5 Å for an ethyl group, 3.6 Å for an ethynyl group, 3.3 Å for a methoxy group, 2.1 Å for a methyl group, and 1.0 Å for a hydrogen atom.

In the formula (1), Z preferably represents a hydrogen atom, a substituted or unsubstituted alkyl group having 2 or less carbon atoms, a substituted or unsubstituted alkynyl group having 2 or less carbon atoms, a substituted or unsubstituted alkenyl group having 2 or less carbon atoms, or a substituted or unsubstituted acyl group having 2 or less carbon atoms, more preferably a hydrogen atom or a substituted or unsubstituted alkyl group having 2 or less carbon atoms, and particularly preferably a hydrogen atom.

In the case where Z represents a substituted alkyl group having 2 or less carbon atoms, examples of the substituent capable of being substituted on the alkyl group include a cyano group, a fluorine atom and a deuterium atom, and a cyano group is preferred. The number of carbon atoms of the substituted alkyl group represented by Z is preferably 1. The substituted or unsubstituted alkyl group having 2 or less carbon atoms represented by Z is preferably a methyl group or an ethyl group, and more preferably a methyl group.

In the case where Z represents a substituted alkynyl group having 2 or less carbon atoms, examples of the substituent capable of being substituted on the alkynyl group include a deuterium atom. Examples of the substituted or unsubstituted alkynyl group having 2 or less carbon atoms represented by Z include an ethynyl group and an ethynyl group substituted by a deuterium atom, and an ethynyl group is preferred.

In the case where Z represents a substituted alkenyl group having 2 or less carbon atoms, examples of the substituent capable of being substituted on the alkenyl group include a deuterium atom. Examples of the substituted or unsubstituted alkenyl group having 2 or less carbon atoms represented by Z include an ethenyl group and an ethenyl group substituted by a deuterium atom, and an ethenyl group is preferred.

In the case where Z represents a substituted acyl group having 2 or less carbon atoms, examples of the substituent capable of being substituted on the acyl group include a fluorine atom. Examples of the substituted or unsubstituted acyl group having 2 or less carbon atoms represented by Z include a formyl group, an acetyl group, and an acetyl group substituted by a fluorine atom, and a formyl group is preferred.

In the formula (1), R¹ to R⁸ each independently represent a hydrogen atom or a substituent, provided that at least one of R¹ to R⁸ represents a substituent represented by the formula (W).

The compound represented by the formula (1) may contain a substituent other than the substituent represented by the formula (W).

Examples of the substituent that may be R¹ to R⁸ in the formula (1) include a halogen atom, an alkyl group (including a cycloalkyl group, a bicycloalkyl group and a tricycloalkyl group), an alkenyl group (including a cycloalkenyl group and a bicycloalkenyl group), an alkynyl group, an aryl group, a heterocyclic group, a cyano group, a hydroxyl group, a nitro group, a carboxyl group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group (including an anilino group), an ammonio group, an acylamino group, an aminocarbonylamino group, an alkoxycarbonylamino group, an aryloxycarbonylamino group, a sulfamoylamino group, an alkyl- or arylsulfonylamino group, a mercapto group, an alkylthio group, an arylthio group, a heterocyclic thio group, a sulfamoyl group, a sulfo group, an alkyl- or arylsulfinyl group, an alkyl- or arylsulfonyl group, an acyl group, an aryloxycarbonyl group, an alkoxycarbonyl group, a carbamoyl group, an aryl- or heterocyclic azo group, an imide group, a phosphino group, a phosphinyl group, a phosphinyloxy group, a phosphinylamino group, a phosphono group, a silyl group, a hydrazino group, a ureido group, a boronic acid group (—B(OH)₂), a phosphato group (—PO(OH)₂), a sulphato group (—OSO₃H), and other known groups.

Among these, a halogen atom, an alkyl group and an aryl group are preferred, and a fluorine atom, an alkyl group having from 1 to 3 carbon atoms and a phenyl group are more preferred.

In the compound represented by the formula (1), the number of the substituent other than the substituent represented by the formula (W) in R¹ to R⁸ is preferably from 0 to 4, more preferably from 0 to 2, and particularly preferably 0.

The substituent represented by the formula (W) will be described.

In the formula (W), L represents a divalent linking group represented by any one of the following formulae (L-1) to (L-12), or a divalent linking group containing 2 or more divalent linking groups each represented by any one of the following formulae (L-1) to (L-12) bonded to each other.

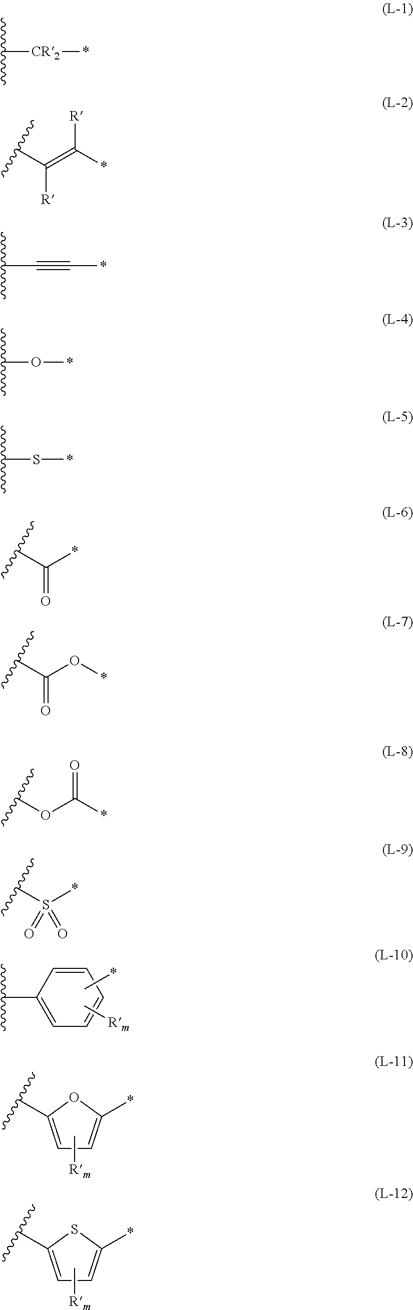

wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

In the case where L represents a divalent linking group containing divalent linking groups each represented by any one of the formulae (L-1) to (L-12) bonded to each other, the number of the divalent linking groups each represented by any one of the formulae (L-1) to (L-12) bonded to each other is preferably from 2 to 4, more preferably 2 or 3, and particularly preferably 2. Particularly in the formulae (L-10) to (L-12), it is also preferred that any one of the formulae (L-1) to (L-12) is further inserted between * and R to form L that represents a linking group containing divalent linking groups each represented by any one of the formulae (L-1) to (L-12) bonded to each other. In the invention, L preferably does not form a divalent linking group containing divalent linking groups each represented by any one of the formulae (L-1) to (L-12) bonded to each other, and thus L preferably represents a divalent linking group represented by any one of the formulae (L-1) to (L-12).

Examples of the substituent R′ in the formulae (L-1), (L-2), (L-10), (L-11) and (L-12) include the groups that are shown as examples of the other substituent that may be R¹ to R⁸ in the formula (1).

In the formula (L-1), R′ each preferably independently represent a hydrogen atom or a substituted or unsubstituted alkyl group having from 1 to 8 carbon atoms, more preferably a substituted or unsubstituted alkyl group having from 1 to 6 carbon atoms, and particularly preferably a substituted or unsubstituted alkyl group having from 1 to 3 carbon atoms. The number of substituent in the two R′ contained in the formula (L-1) is preferably 0 or 1, and particularly preferably 0. In the case where the four R′ contained in the formula (L-1) have a substituent, the carbon atom adjacent to * in the formula (L-1) preferably has a substituent.

In the formula (L-2), R′ each preferably independently represent a hydrogen atom or a substituted or unsubstituted alkyl group having 1 or more carbon atoms, more preferably a hydrogen atom or a substituted or unsubstituted alkyl group having from 1 to 4 carbon atoms, and particularly preferably a hydrogen atom.

In the formulae (L-10), (L-11) and (L-12), R′ each preferably independently represent a hydrogen atom, a substituted or unsubstituted alkyl group having 1 or more carbon atoms, a substituted or unsubstituted alkenyl group having 2 or more carbon atoms, a substituted or unsubstituted alkynyl group having 2 or more carbon atoms, or a substituted or unsubstituted alkoxy group having 1 or more carbon atoms, more preferably a hydrogen atom, a substituted or unsubstituted alkyl group having from 1 to 12 carbon atoms, a substituted or unsubstituted alkenyl group having from 2 to 12 carbon atoms, a substituted or unsubstituted alkynyl group having from 2 to 12 carbon atoms, or a substituted or unsubstituted alkoxy group having from 1 to 12 carbon atoms, and particularly preferably a hydrogen atom.

In the formula (L-10), m represents 4; and in the formulae (L-11) and (L-12), m represents 2.

L preferably represents a divalent linking group represented by any one of the formulae (L-1) to (L-4), (L-6), (L-10), (L-11) and (L-12), or a divalent linking group containing 2 or more of the divalent linking groups bonded to each other, more preferably a divalent linking group represented by any one of the formulae (L-1) to (L-4), (L-6), (L-10) and (L-12), or a divalent linking group containing 2 or more of the divalent linking groups bonded to each other, particularly preferably a divalent linking group represented by any one of the formulae (L-1) to (L-3), (L-10), (L-11) and (L-12) or a divalent linking group containing 2 or more of the divalent linking groups bonded to each other, further particularly preferably a divalent linking group represented by the formula (L-1) or (L-10) or a divalent linking group containing 2 or more of the divalent linking groups bonded to each other from the standpoint of the carrier transport property, and still further particularly preferably a divalent linking group represented by the formula (L-1) or (L-10) from the standpoint of the chemical stability and the carrier transport property.

In the formula (W), R represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R has a number of carbon atoms of 2 or more in the case where L represents a divalent linking group represented by any one of the formulae (L-1) to (L-3), or 4 or more in the case where L represents a divalent linking group represented by any one of the formulae (L-4) to (L-12). In the formula (W), R preferably represents a substituted or unsubstituted alkyl group. R may represent a substituted or unsubstituted trialkylsilyl group only in the case where L adjacent to R represents a divalent linking group represented by the formula (L-3).

In the formula (W), in the case where R represents a substituted or unsubstituted alkyl group, and L represents a divalent linking group represented by the formula (L-1), the number of carbon atoms of the alkyl group is 2 or more, preferably from 2 to 12, and more preferably from 3 to 10 from the standpoint of the enhancement of the carrier mobility.

In the formula (W), in the case where R represents a substituted or unsubstituted alkyl group, and L represents a divalent linking group represented by the formula (L-2) or (L-3), the number of carbon atoms of the alkyl group is 2 or more, preferably from 2 to 12, more preferably from 3 to 10 from the standpoint of the enhancement of the carrier mobility, and particularly preferably from 4 to 9 from the standpoint of the further enhancement of the carrier mobility.

In the formula (W), in the case where R represents a substituted or unsubstituted alkyl group, and L represents a divalent linking group represented by any one of the formulae (L-4) to (L-12), the number of carbon atoms of the alkyl group is 4 or more, preferably from 4 to 12, and more preferably from 6 to 12 from the standpoint of the enhancement of the carrier mobility.

In the case where L represents a divalent linking group containing 2 or more of the divalent linking groups represented by any one of the formulae (L-1) to (L-12), the preferred range of the number of carbon atoms of the substituted or unsubstituted alkyl group represented by R is determined by the kind of the formulae (L-1) to (L-12) that is adjacent to R.

In the compound represented by the formula (1), in the case where the group represented by the formula (W) contains an alkyl group, the carrier mobility may be enhanced when the total number of carbon atoms of the alkyl groups contained in L and R is 4 or more, and the carrier mobility may be further enhanced when the total number of carbon atoms in the main chains of the alkyl groups contained in L and R is 4 or more.

The alkyl group that may be R may be any one of linear, branched and cyclic, and is preferably a linear alkyl group. In the case where R represents an alkyl group having a substituent, examples of the substituent include a halogen atom, and a fluorine atom is preferred. In the case where R represents an alkyl group having a fluorine atom, the alkyl group may be a perfluoroalkyl group, in which all the hydrogen atoms of the alkyl group are replaced by fluorine atoms.

In the case where R in the formula (W) represents an oligooxyethylene group having a repeating number of an oxyethylene group of 2 or more, the oligooxyethylene group represented by R herein means a group represented by —(CH₂CH₂)ᵥOY (wherein the repeating number v of an oxyethylene unit is an integer of 2 or more, and Y as the terminal group represents a hydrogen atom or a substituent). In the case where Y as the terminal group of the oligooxyethylene group is a hydrogen atom, the group is a hydroxyl group. The repeating number v of an oxyethylene unit is preferably from 2 to 4, and more preferably from 2 to 3. The terminal hydroxyl group of the oligooxyethylene group is preferably blocked, i.e., Y preferably represents a substituent. In this case, the hydroxyl group is preferably blocked with an alkyl group having from 1 to 3 carbon atoms, i.e., Y preferably represents an alkyl group having from 1 to 3 carbon atoms, and Y more preferably represents a methyl group or an ethyl group, and particularly preferably a methyl group.

In the case where R in the formula (W) represents an oligosiloxane group having 2 or more silicon atoms, the repeating number of a siloxane unit is preferably from 2 to 4, and more preferably from 2 to 3. The Si atom is preferably bonded to a hydrogen atom or an alkyl group. In the case where the Si atom is bonded to an alkyl group, the number of carbon atoms of the alkyl group is preferably from 1 to 3, and for example, a methyl group or an ethyl group is preferably bonded thereto. The Si atom may be bonded to the same alkyl groups or may be bonded to different alkyl groups or a hydrogen atom. The siloxane units constituting the oligosiloxane group may be all the same as each other or different from each other, and are preferably all the same as each other.

In the formula (W), only in the case where L adjacent to R represents a divalent linking group represented by the formula (L-3), R may represent a substituted or unsubstituted trialkylsilyl group. In the case where R in the formula (W) represents a substituted or unsubstituted trialkylsilyl group, the alkyl group bonded to the Si atom preferably has from 1 to 3 carbon atoms, and for example, a methyl group, an ethyl group, an isopropyl group or the like is preferably bonded to the Si atom. The alkyl groups bonded to the Si atom may be the same as or different from each other. In the case where R represents a trialkylsilyl group having a substituent, the substituent is not particularly limited.

In the compound represented by the formula (1), the number of the substituent that is represented by the formula (W) in R¹ to R⁸ is preferably from 1 to 4, and more preferably from 1 to 2.

In the formula (1) in the invention, at least one of R², R³, R⁶ and R⁷ preferably represents a substituent represented by the formula (W) from the standpoint of the achievement of both the solubility and the carrier mobility. Furthermore, one or two positions of any one of R² and R³ and any one of R⁶ and R⁷ are more preferably substituted from the standpoint of the achievement of both the solubility and the carrier mobility.

It is considered that the reason why these positions are preferred as the substitution positions in the formula (1) is that the compound is excellent in chemical stability and is preferred also from the standpoint of the HOMO level and the molecular packing in the film. In particular, when 1 or 2 positions of any one of R² and R³ and any one of R⁶ and R⁷ each represent a substituent, a high carrier concentration may be obtained.

In the invention, the compound represented by the formula (1) is preferably a compound represented by the following formula (2-1), (2-2) or (3) from the standpoint of the enhancement of the carrier mobility while enhancing the solubility.

In the following description, the preferred ranges of the formula (2-1), the formula (2-2) and the formula (3) will be described in this order. In the formulae (L-1) to (L-12) in the description for the formulae (2-1), (2-2) and (3), * each independently represent a position bonded to anyone of R^{a}, R^{b}, R^{c} and R^{d} adjacent to the formulae (L-1) to (L-12).

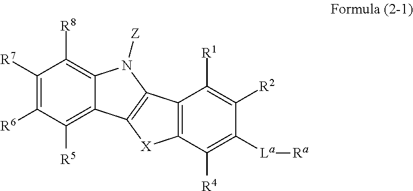

wherein in the formula (2-1), X represents a S atom or an O atom; Z represents a substituent that has a length of 3.7 Å or less from the N atom to the end of the substituent; R¹, R² and R⁴ to R⁸ each independently represent a hydrogen atom or a substituent, provided that R⁷ is not a substituent represented by -L^{a}-R^{a}; L^{a} represents a divalent linking group represented by any one of the following formulae (L-1) to (L-12), or a divalent linking group containing 2 or more divalent linking groups each represented by any one of the following formulae (L-1) to (L-12) bonded to each other; and R^{a} represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R^{a} has a number of carbon atoms of 2 or more in the case where L^{a} represents a divalent linking group represented by any one of the formulae (L-1) to (L-3), or 4 or more in the case where L^{a} represents a divalent linking group represented by any one of the formulae (L-4) to (L-12), and R^{a} represents a substituted or unsubstituted trialkylsilyl group only in the case where L^{a} adjacent to R^{a} represents a divalent linking group represented by the formula (L-3).

In the formula (2-1), the preferred ranges of Z are the same as the preferred ranges of Z in the formula (1).

In the formula (2-1), R¹, R² and R⁴ to R⁸ each independently represent a hydrogen atom or a substituent, provided that R⁷ is not a substituent represented by -L^{a}-R^{a}. In the formula (2-1), the preferred ranges of the substituents represented by R¹, R² and R⁴ to R⁸ are the same as the preferred ranges of the substituents represented by R¹ to R⁸ in the formula (1) other than the substituent represented by the formula (W).

In the formula (2-1), the preferred ranges of L^{a} are the same as the preferred ranges of L in the formula (W).

In the formula (2-1), the preferred ranges of R^{a} are the same as the preferred ranges of R in the formula (W).

Among these, in the formula (2-1), it is preferred that R^{a} represents a branched alkyl group; or L^{a} represents a divalent linking group represented by the formula (L-1), and at least one of R′ in the divalent linking groups represented by the formula (L-1) represents an alkyl group since the compounds represented by the formula (2-1) undergo favorable molecular packing (i.e., the orientation of the plural molecules is promoted by aligning the adjacent molecules directed oppositely to each other), and the crystallinity of the herringbone structure may be enhanced.

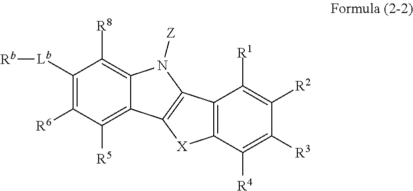

wherein in the formula (2-2), X represents a S atom or an O atom; Z represents a substituent that has a length of 3.7 Å or less from the N atom to the end of the substituent; R¹ to R⁶ and R⁸ each independently represent a hydrogen atom or a substituent, provided that R³ is not a substituent represented by -L^{b}-R^{b}; L^{b} represents a divalent linking group represented by any one of the following formulae (L-1) to (L-12), or a divalent linking group containing 2 or more divalent linking groups each represented by any one of the following formulae (L-1) to (L-12) bonded to each other; and R^{b} represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R^{b} has a number of carbon atoms of 2 or more in the case where L^{b} represents a divalent linking group represented by any one of the formulae (L-1) to (L-3), or 4 or more in the case where L^{b} represents a divalent linking group represented by any one of the formulae (L-4) to (L-12), and R^{b} represents a substituted or unsubstituted trialkylsilyl group only in the case where L^{b} adjacent to R^{b} represents a divalent linking group represented by the formula (L-3).

In the formula (2-2), the preferred ranges of Z are the same as the preferred ranges of Z in the formula (1).

In the formula (2-2), R¹ to R⁶ and R⁸ each independently represent a hydrogen atom or a substituent, provided that R³ is not a substituent represented by -L^{b}-R^{b}. In the formula (2-2), the preferred ranges of the substituents represented by R¹ to R⁶ and R⁸ are the same as the preferred ranges of the substituents represented by R¹ to R⁸ in the formula (1) other than the substituent represented by the formula (W).

In the formula (2-2), the preferred ranges of L^{b} are the same as the preferred ranges of L in the formula (W).

In the formula (2-2), the preferred ranges of R^{b} are the same as the preferred ranges of R in the formula (W).

Among these, in the formula (2-2), it is preferred that R^{b} represents a branched alkyl group; or L^{b} represents a divalent linking group represented by the formula (L-1), and at least one of R′ in the divalent linking groups represented by the formula (L-1) represents an alkyl group since the compounds represented by the formula (2-2) undergo favorable molecular packing (i.e., the orientation of the plural molecules is promoted by aligning the adjacent molecules directed oppositely to each other), and the crystallinity of the herringbone structure may be enhanced.

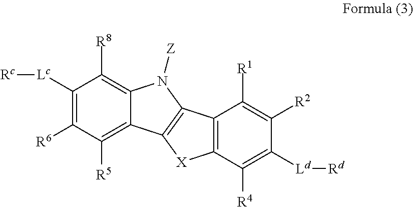

wherein in the formula (3), X represents a S atom or an O atom; Z represents a substituent that has a length of 3.7 Å or less from the N atom to the end of the substituent; R¹, R², R⁴ to R⁶ and R⁸ each independently represent a hydrogen atom or a substituent; L^{c} and L^{d} each independently represent a divalent linking group represented by any one of the following formulae (L-1) to (L-12), or a divalent linking group containing 2 or more divalent linking groups each represented by any one of the following formulae (L-1) to (L-12) bonded to each other; and R^{c} and R^{d} each independently represent a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl groups represented by R^{c} and R^{d} each have a number of carbon atoms of 2 or more in the case where L^{c} and L^{d} respectively represent a divalent linking group represented by any one of the formulae (L-1) to (L-3), or 4 or more in the case where L^{c} and L^{d} respectively represent a divalent linking group represented by any one of the formulae (L-4) to (L-12), and R^{c} and R^{d} each represent a substituted or unsubstituted trialkylsilyl group only in the case where L^{c} and L^{d} adjacent to R^{c} and R^{d} respectively represent a divalent linking group represented by the formula (L-3).

In the formula (3), the preferred ranges of Z are the same as the preferred ranges of Z in the formula (1).

In the formula (3), the preferred ranges of the substituents represented by R¹, R², R⁴ to R⁶ and R⁸ are the same as the preferred ranges of the substituents represented by R¹ to R⁸ in the formula (1) other than the substituent represented by the formula (W).

In the formula (3), the preferred ranges of L^{c} and L^{d} are the same as the preferred ranges of L in the formula (W), and L^{c} and L^{d} may be the same as or different from each other, and are preferably the same as each other.

In the formula (3), the preferred ranges of R^{c} and R^{d} are the same as the preferred ranges of R in the formula (W), and R^{c} and R^{d} may be the same as or different from each other, and are preferably the same as each other.

Among these, in the formula (3), it is preferred that R^{c} and R^{d} each independently represent a linear alkyl group, provided that in the case where L^{c} and L^{d} each independently represent a divalent linking group represented by the formula (L-1), all R′ in the divalent linking groups represented by the formula (L-1) each represent a hydrogen atom, since the compounds represented by the formula (3) undergo favorable molecular packing (i.e., the orientation of the plural molecules are promoted by aligning the adjacent molecules directed oppositely to each other), and the crystallinity of the herringbone structure may be enhanced.

Specific examples of the compound represented by the formula (1) are shown below, but the compound represented by the formula (1) capable of being used in the invention is not construed as being limited to the specific examples.

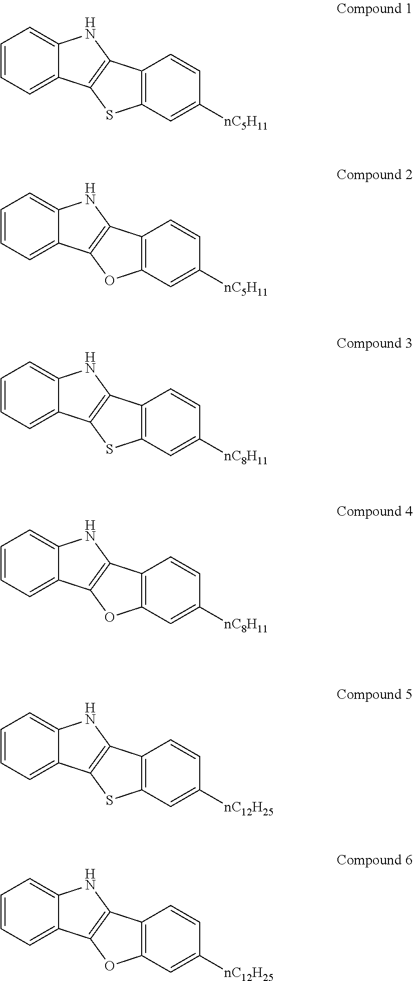

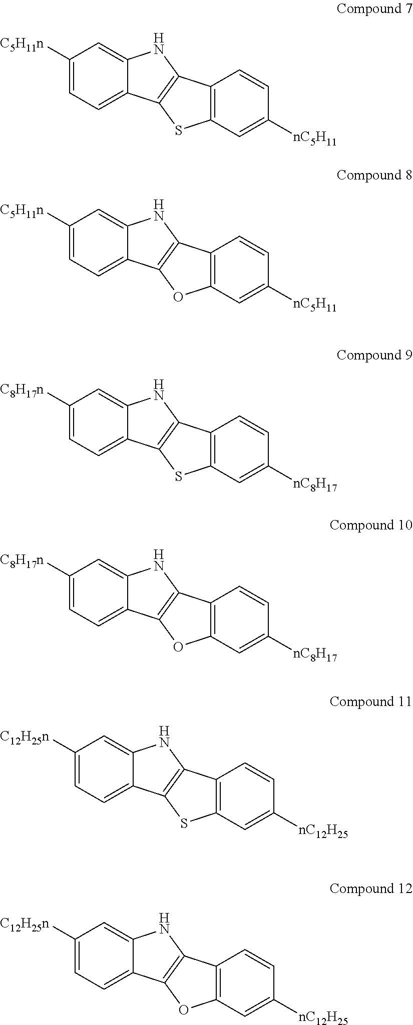

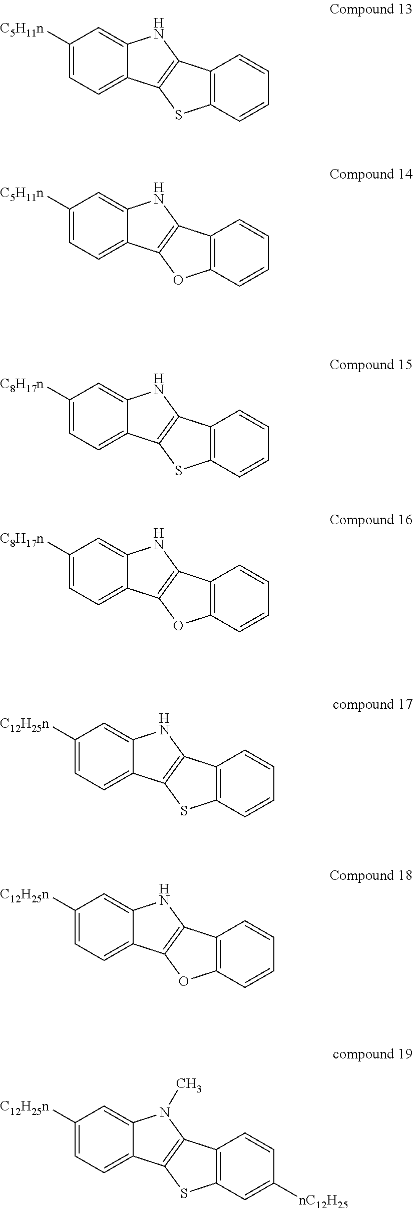

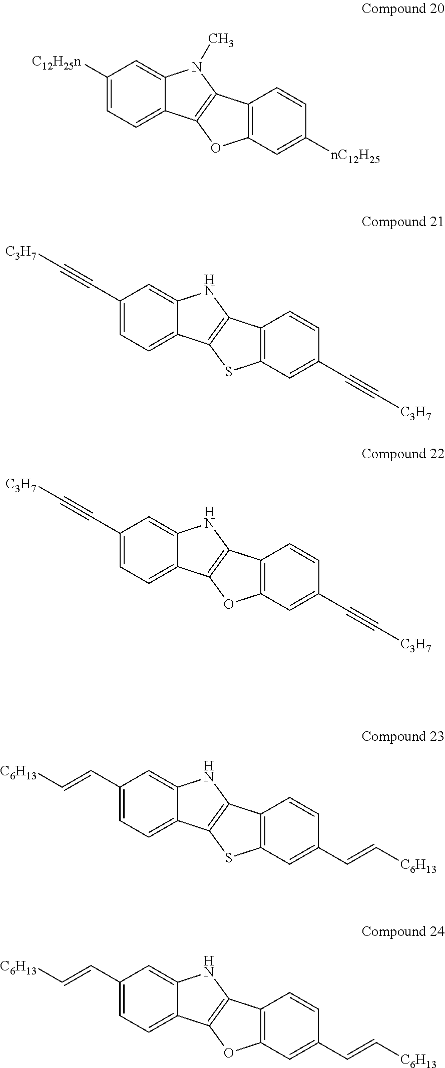

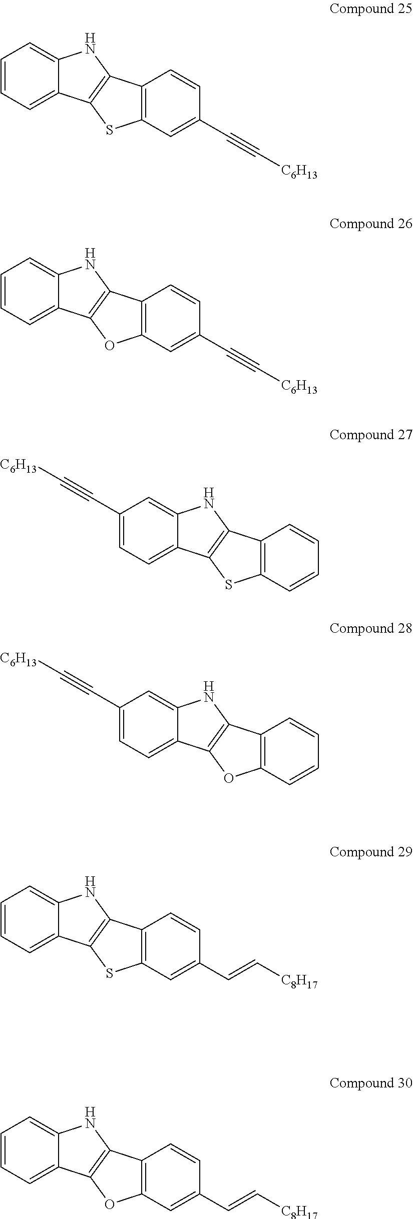

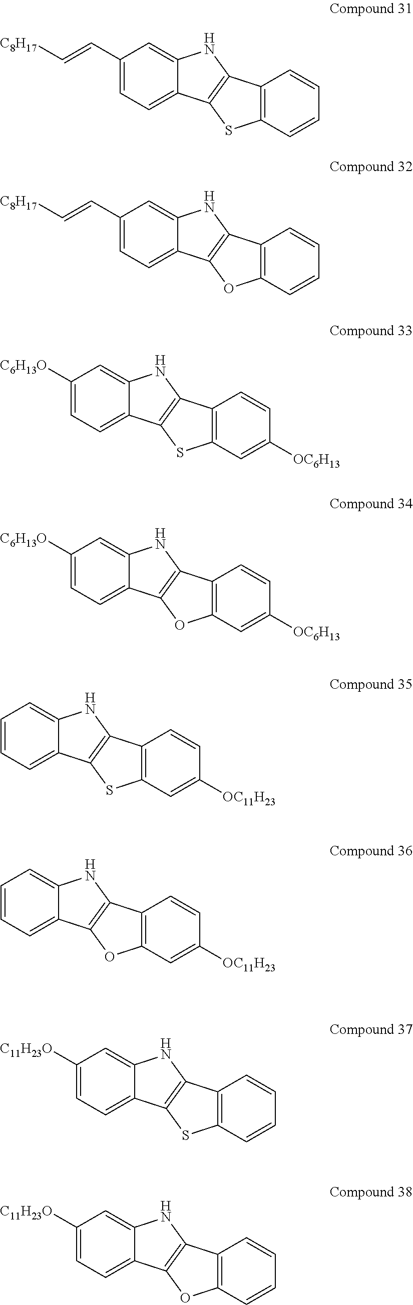

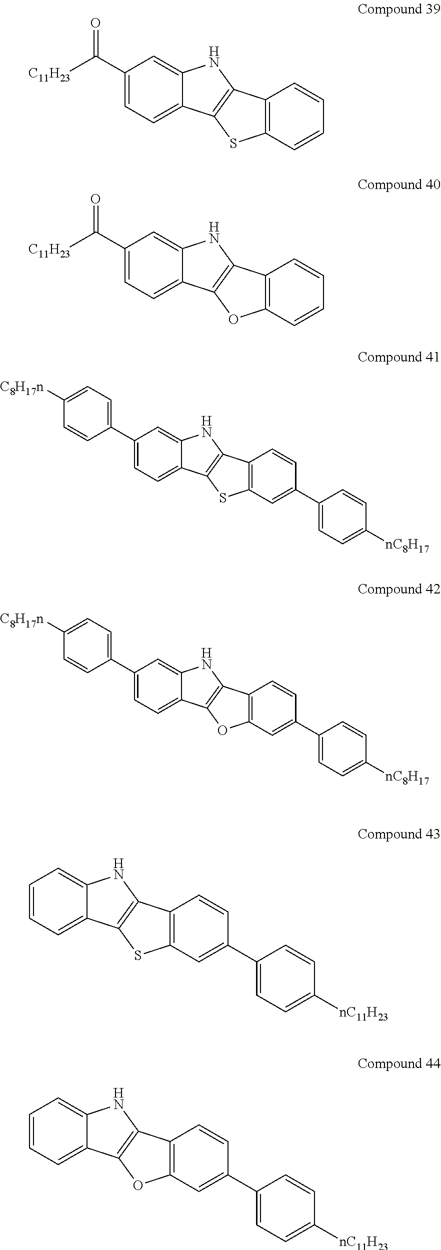

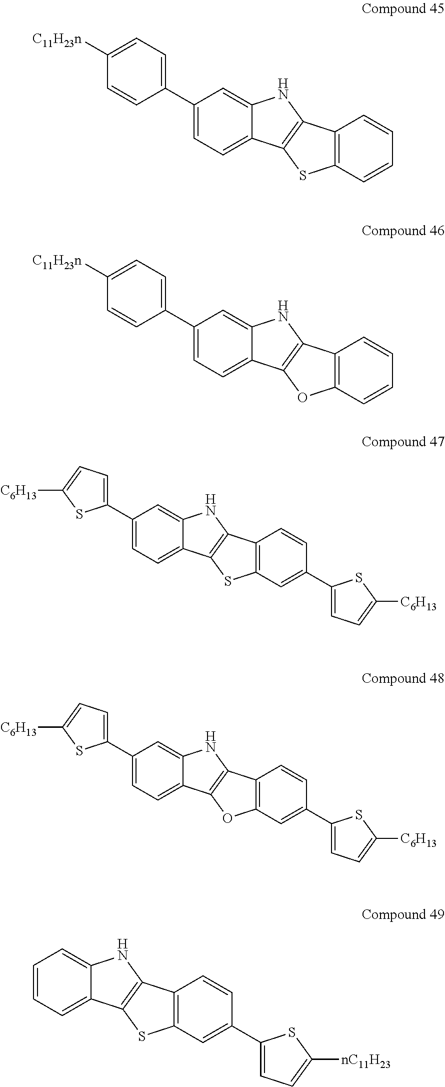

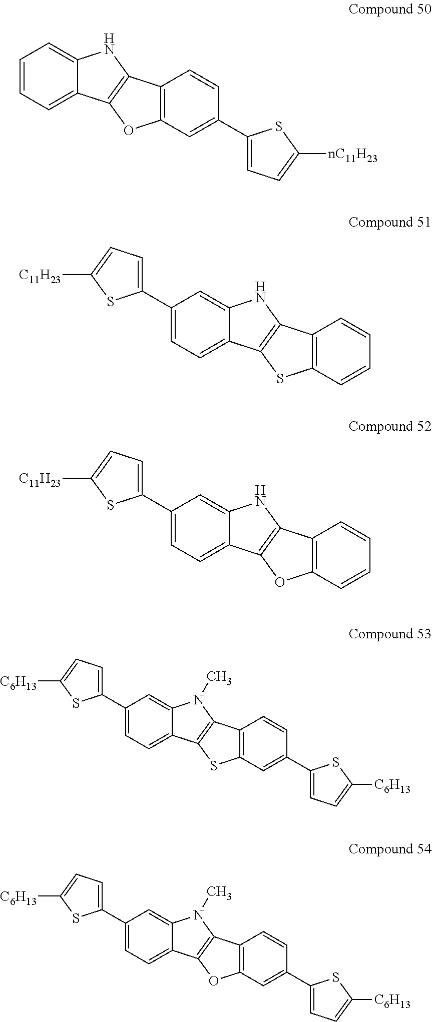

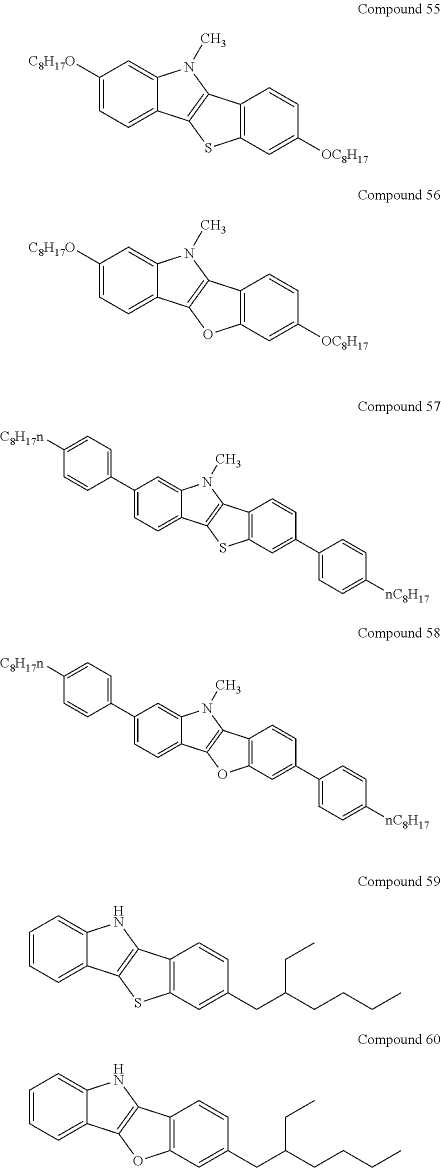

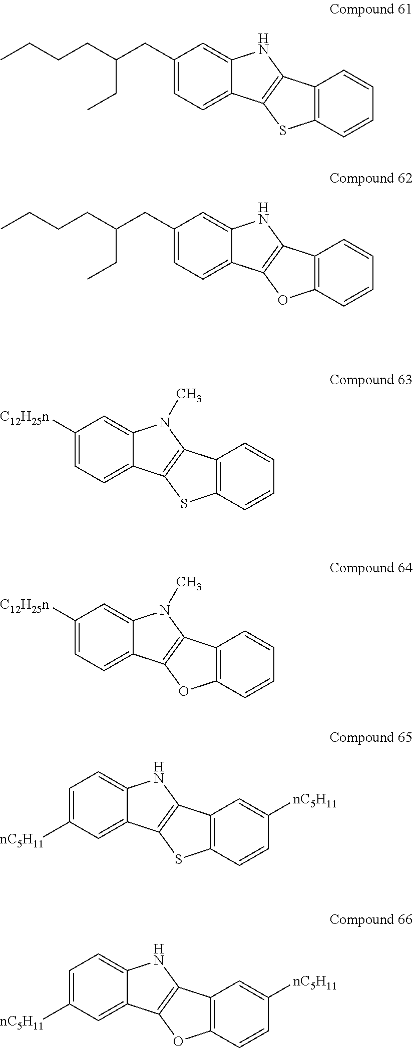

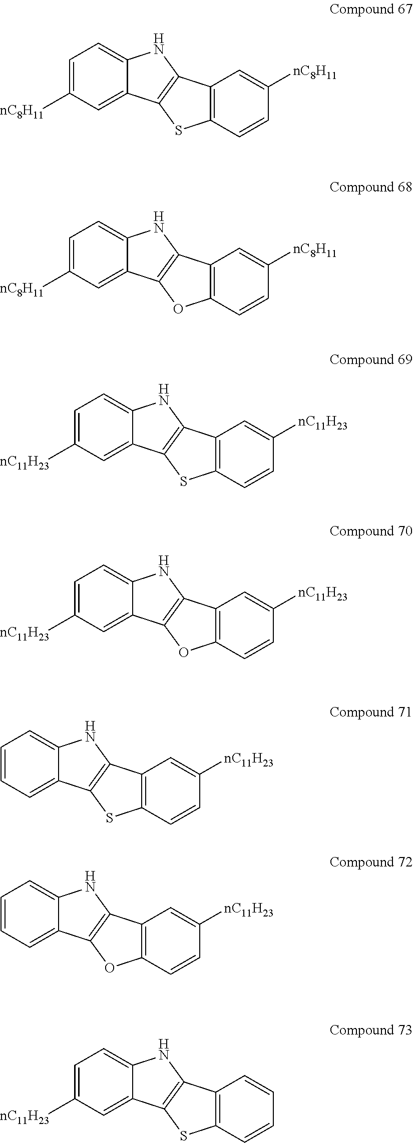

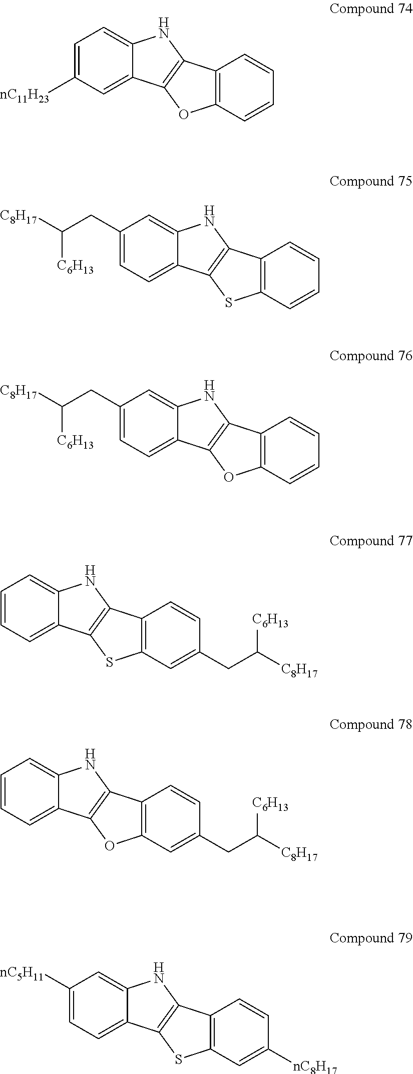

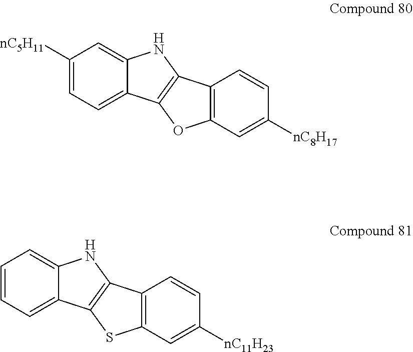

The compound represented by the formula (1) preferably has a molecular weight of 3,000 or less, more preferably 2,000 or less, further preferably 1,000 or less, and particularly preferably 850 or less. The molecular weight that is the upper limit or less is preferred since the compound has increased solubility in a solvent.

The molecular weight of the compound is preferably 400 or more, more preferably 450 or more, and further preferably 500 or more, from the standpoint of the stability of the film quality of the thin film.

The compound represented by the formula (1) may be synthesized by combining the methods described in JP-A-2010-270084, JP-A-2009-182034, JP-A-2010-177644 and WO 2012/035934 and the known reactions.

In the reaction of forming the benzothienoindole ring or the benzofuranoindole ring, any reaction condition may be used. The reaction solvent used may be any solvent. An acid or a base is preferably used for promoting the ring-forming reaction, and particularly a base is preferably used. The optimum reaction condition may vary depending on the structure of the target benzothienoindole or benzofuranoindole derivative, and may be determined with reference to the specific reaction shown in the aforementioned literature.

The synthesis intermediates having the various substituents may be synthesized by combining known reactions. The substituents may be introduced in any stage of the intermediates. The intermediates after synthesis is preferably purified by column chromatography, recrystallization or the like, and then purified by sublimation. The sublimation purification not only isolates organic impurities, but also effectively removes an inorganic salt, a residual solvent and the like.

### Structure of Organic Thin Film Transistor

The organic thin film transistor of the invention contains the compound represented by the formula (1) in a semiconductor active layer.

The organic thin film transistor of the invention may further contain other layers in addition to the semiconductor active layer.

The organic thin film transistor of the invention is preferably used as an organic field effect transistor (FET), and is more preferably used as an insulated gate FET, in which the gate and the channel are insulated from each other.

Preferred embodiments of the organic thin film transistor of the invention will be described below with reference to the drawings, but the invention is not limited to the embodiments.

### Laminated Structure

The laminated structure of the organic field effect transistor is not particularly limited, and various known structures may be used.

One example of the structure of the organic thin film transistor of the invention is a bottom-gate top-contact structure having a substrate as the lowermost layer having disposed thereon an electrode, an insulating layer, a semiconductor active layer (organic semiconductor layer), and two electrodes, in this order. In this structure, the electrode on the upper surface of the substrate as the lowermost layer is provided on a part of the substrate, and the insulating layer is disposed to be in contact with the substrate in the portion other than the electrode. The two electrodes disposed on the upper surface of the semiconductor active layer are disposed to be separated from each other.

A structure of a bottom-gate top-contact device is shown in FIG. 1. FIG. 1 is a schematic illustration showing a cross sectional structure of one example of the organic thin film transistor of the invention. The organic thin film transistor shown in FIG. 1 has a substrate 11 disposed as the lowermost layer, an electrode 12 disposed on a part of the upper surface of the substrate 11, and an insulating layer 13 disposed to cover the electrode 12 and to be in contact with the substrate 11 in the portion other than the electrode 12. A semiconductor active layer 14 is provided on the upper surface of the insulating layer 13, and two electrodes 15a and 15b, which are separated from each other, are disposed on parts of the semiconductor active layer 14.

In the organic thin film transistor shown in FIG. 1, the electrode 12 is a gate, and the electrodes 15a and 15b each are a drain or a source. The organic thin film transistor shown in FIG. 1 is an insulated gate FET, in which the channel, which is an electric current path between the drain and the source, and the gate are insulated from each other.

Another example of the structure of the organic thin film transistor of the invention is a bottom-gate bottom-contact device.

A structure of a bottom-gate bottom-contact device is shown in FIG. 2. FIG. 2 is a schematic illustration showing a cross sectional structure of an organic thin film transistor that is produced as a substrate for measuring FET characteristics in the example of the invention. The organic thin film transistor shown in FIG. 2 has a substrate 31 disposed as the lowermost layer, an electrode 32 disposed on a part of the upper surface of the substrate 31, and an insulating layer 33 disposed to cover the electrode 32 and to be in contact with the substrate 31 in the portion other than the electrode 32. A semiconductor active layer 35 is provided on the upper surface of the insulating layer 33, and two electrodes 34a and 34b are disposed under the semiconductor active layer 35.

In the organic thin film transistor shown in FIG. 2, the electrode 32 is a gate, and the electrodes 34a and 34b each are a drain or a source. The organic thin film transistor shown in FIG. 2 is an insulated gate FET, in which the channel, which is an electric current path between the drain and the source, and the gate are insulated from each other.

Other preferred examples of the structure of the organic thin film transistor of the invention include a top-gate top-contact device and a top-gate bottom-contact device, in which an insulator and a gate electrode are disposed on an organic semiconductor layer.

### Thickness

The organic thin film transistor of the invention preferably has a total thickness of the transistor, for example, of from 0.1 to 0.5 μm, in the case where a thinner transistor is demanded.

### Sealing

For shielding the organic thin film transistor device from the air and water to enhance the storage stability of the organic thin film transistor device, the entire organic thin film transistor device may be sealed with a metallic sealing canister, an inorganic material, such as glass and silicon nitride, a polymer material, such as parylene, a low molecular weight material, and the like.

Preferred embodiments of the layers of the organic thin film transistor of the invention will be described below, but the invention is not limited to the embodiments.

### Substrate

### Material

The organic thin film transistor of the invention preferably contains a substrate.

The material for the substrate is not particularly limited, and known materials may be used. Examples of the material include a polyester film, such as polyethylene naphthoate (PEN) and polyethylene terephthalate (PET), a cycloolefin polymer film, a polycarbonate film, a triacetyl cellulose (TAC) film, a polyimide film, these polymer films having an ultrathin glass layer laminated thereon, ceramics, silicone, quartz, glass, and the like, and silicone is preferred.

### Electrode

### Material

The organic thin film transistor of the invention preferably contains an electrode.

Examples of the material for the electrode include known electroconductive materials, for example, a metal material, such as Cr, Al, Ta, Mo, Nb, Cu, Ag, Au, Pt, Pd, In, Ni and Nd, an alloy material of the metal materials, a carbon material, and an electroconductive polymer, which may be used without particular limitation.

### Thickness

The thickness of the electrode is not particularly limited and is preferably from 10 to 50 nm.

The gate width (or the channel width) W and the gate length (or the channel length) L are not particularly limited, and the ratio W/L is preferably 10 or more, and more preferably 20 or more.

### Insulating Layer

### Material

The material for the insulating layer is not particularly limited as far as the necessary insulating effect is obtained, and examples thereof include silicon dioxide, silicon nitride, a fluorine polymer insulating material, such as PTFE and CYTOP, a polyester insulating material, a polycarbonate insulating material, an acrylic polymer insulating material, an epoxy resin insulating material, a polyimide insulating material, a polyvinylphenol resin insulating material, and poly-p-xylylene resin insulating material.

The upper surface of the insulating layer may be surface-treated, and preferred examples thereof used include an insulating layer formed of silicon dioxide, the surface of which is surface-treated by coating hexamethyldisilazane (HMDS) or octadecyltrichlorosilane (OTS) thereon.

### Thickness

The thickness of the insulating layer is not particularly limited, and in the case where a thin insulating layer is demanded, the thickness thereof is preferably from 10 to 400 nm, more preferably from 20 to 200 nm, and particularly preferably from 50 to 200 nm.

### Semiconductor Active Layer

### Material

The organic thin film transistor of the invention contains the compound represented by the formula (1), i.e., the compound of the invention, in the semiconductor active layer.

The semiconductor active layer may be a layer that is formed of the compound of the invention, or a layer containing a polymer binder described later in addition to the compound of the invention. The semiconductor active layer may contain a residual solvent used on forming the film.

The content of the polymer binder in the semiconductor active layer is not particularly limited, and the polymer binder is preferably used in a range of from 0 to 95% by mass, more preferably used in a range of from 10 to 90% by mass, further preferably used in a range of from 20 to 80% by mass, and particularly preferably used in a range of from 30 to 70% by mass.

### Thickness

The thickness of the semiconductor active layer is not particularly limited, and in the case where a thin semiconductor active layer is demanded, the thickness thereof is preferably from 10 to 400 nm, more preferably from 10 to 200 nm, and particularly preferably from 10 to 100 nm.

### Organic Semiconductor Material for Non-Light Emitting Organic Semiconductor Device

The invention also relates to an organic semiconductor material for a non-light emitting organic semiconductor device containing the compound represented by the formula (1), i.e., the compound of the invention.

### Non-Light Emitting Organic Semiconductor Device

The non-light emitting organic semiconductor device referred herein means a device that is not intended to emit light. The non-light emitting organic semiconductor device is preferably a non-light emitting organic semiconductor device that uses an electronic element having a layer structure of thin films. The non-light emitting organic semiconductor device encompasses an organic thin film transistor, an organic photoelectric conversion device (such as a solid state imaging device for a photosensor, and a solar cell for energy conversion), a gas sensor, an organic rectifying device, an organic inverter, an information recording device, and the like. The organic photoelectric conversion device may be used for both a photosensor (i.e., a solid state imaging device) and energy conversion (i.e., a solar cell). Preferred examples of the device include an organic photoelectric conversion device and an organic thin film transistor, and more preferred examples thereof include an organic thin film transistor. Accordingly, the organic semiconductor material for a non-light emitting organic semiconductor device of the invention is preferably a material for an organic thin film transistor as described above.

### Organic Semiconductor Material

The organic semiconductor material referred herein means an organic material that shows characteristics of a semiconductor. The organic semiconductor material includes a p-type (hole transporting) organic semiconductor, which shows conductivity with holes as a carrier, and an n-type (electron transporting) organic semiconductor, which shows conductivity with electrons as a carrier, as similar to a semiconductor material formed of an inorganic material.

The compound of the invention may be used as any of a p-type organic semiconductor material and an n-type organic semiconductor material, and is preferably used as a p-type organic semiconductor material. The flowability of a carrier in an organic semiconductor is shown by a carrier mobility μ. The carrier mobility μ is preferably as large as possible, and is preferably 5×10⁻³ cm²/Vs or more, more preferably 1×10⁻² cm²/Vs or more, particularly preferably 5×10⁻² cm²/Vs or more, further particularly preferably 1×10⁻¹ cm²/Vs or more, still further particularly preferably 5×10⁻¹ cm²/Vs or more, and still further particularly preferably 1 cm²/Vs or more. The carrier mobility μ may be obtained from the characteristics of a field effect transistor (FET) device produced or by a time-of-flight (TOF) measurement method.

### Organic Semiconductor Thin Film for Non-Light Emitting Organic Semiconductor Device

### Material

The invention also relates to an organic semiconductor thin film for a non-light emitting organic semiconductor device containing the compound represented by the formula (1), i.e., the compound of the invention.

The organic semiconductor thin film for a non-light emitting organic semiconductor device of the invention contains the compound represented by the formula (1), i.e., the compound of the invention, and an embodiment thereof that contains no polymer binder is also preferred.

The organic semiconductor thin film for a non-light emitting organic semiconductor device of the invention may contain the compound represented by the formula (1), i.e., the compound of the invention, and a polymer binder.

Examples of the polymer binder include an insulating polymer, such as polystyrene, polycarbonate, polyarylate, polyester, polyamide, polyimide, polyurethane, polysiloxane, polysulfone, polymethyl methacrylate, polymethyl acrylate, cellulose, polyethylene and polypropylene, copolymers thereof, a photoconductive polymer, such as polyvinylcarbazole and polysilane, and an electroconductive polymer and a semiconductor polymer, such as polythiophene, polypyrrole, polyaniline and poly-p-phenylenevinylene.

The polymer binder may be used solely or as a combination of plural kinds thereof.

The organic semiconductor material and the polymer binder may be uniformly mixed, or a part or the whole thereof may be phase-separated, and from the standpoint of the charge mobility, such a structure that the organic semiconductor and the binder are phase-separated in the thickness direction in the film is most preferred since the charge migration of the organic semiconductor may not be inhibited by the binder.

Taking the mechanical strength of the thin film into consideration, a polymer binder having a high glass transition temperature is preferred, and taking the charge mobility into consideration, a polymer binder having a structure that contains no polar group, a photoconductive polymer, and an electroconductive polymer are preferred.

The amount of the polymer binder used is not particularly limited, and the polymer binder may be preferably used in a range of from 0 to 95% by mass, more preferably used in a range of from 10 to 90% by mass, further preferably used in a range of from 20 to 80% by mass, and particularly preferably used in a range of from 30 to 70% by mass, in the organic semiconductor thin film for a non-light emitting organic semiconductor device of the invention.

In the invention, an organic thin film having good film quality may be obtained by using the compound having the aforementioned structure. Specifically, the compound of the invention has good crystallinity to enable formation of a film having a sufficient thickness, and thus the organic semiconductor thin film for a non-light emitting organic semiconductor device of the invention thus obtained may have good quality.

### Film Forming Method

The compound of the invention may be formed as a film on a substrate by any method.

On forming the film, the substrate may be heated or cooled, and the film quality and the molecular packing in the film may be controlled by changing the temperature of the substrate. The temperature of the substrate is not particularly limited, and is preferably in a range of from 0 to 200° C., more preferably in a range of from 15 to 100° C., and particularly preferably in a range of from 20 to 95° C.

On forming a film of the compound of the invention on a substrate, the film may be formed by a vacuum process or a solution process, both of which are preferred.

Specific examples of the film formation by a vacuum process include a physical vapor phase growing method, such as a vacuum vapor deposition method, a sputtering method, an ion plating method and a molecular beam epitaxy (MBE) method, and a chemical vapor deposition (CVD) method, such as plasma polymerization, and a vacuum vapor deposition method is preferably used.

The film formation by a solution process means a method, in which an organic compound is dissolved in a solvent capable of dissolving the same, and a film is formed by using the resulting solution. Specific examples thereof used include ordinary methods, for example, a coating method, such as a casting method, a dip coating method, a die coater method, a roll coater method, a bar coater method and a spin coating method, a printing method, such as an ink-jet method, a screen printing method, a gravure printing method, a flexography printing method, an offset printing method and a microcontact printing method, and a Langmuir-Blodgett (LB) method, and a casting method, a spin coating method, an ink-jet method, a gravure printing method, a flexography printing method, an offset printing method and a microcontact printing method are particularly preferably used.

The organic semiconductor thin film for a non-light emitting organic semiconductor device of the invention is preferably produced by a solution coating method. In the case where the organic semiconductor thin film for a non-light emitting organic semiconductor device of the invention contains a polymer binder, the thin film is preferably formed such a method that the material for forming the layer and the polymer binder are dissolved or dispersed in a suitable solvent to prepare a coating liquid, which is then coated by various coating methods to form the thin film.

The coating solution for a non-light emitting organic semiconductor device of the invention capable of being used for film formation by a solution process will be described below.

### Coating Solution for Non-Light Emitting Organic Semiconductor Device

The invention also relates to a coating solution for a non-light emitting organic semiconductor device containing the compound represented by the formula (1), i.e., the compound of the invention.

In the case where the film is formed on a substrate by a solution process, the material for forming the layer may be dissolved or dispersed in a suitable organic solvent (for example, a hydrocarbon solvent, such as hexane, octane, decane, toluene, xylene, mesitylene, ethylbenzene, decalin and 1-methylnaphthalene, a ketone solvent, such as acetone, methyl ethyl ketone, methyl isobutyl ketone and cyclohexanone, a halogenated hydrocarbon solvent, such as dichloromethane, chloroform, tetrachloromethane, dichloroethane, trichloroethane, tetrachloroethane, chlorobenzene, dichlorobenzene and chlorotoluene, an ester solvent, such as ethyl acetate, butyl acetate and amyl acetate, an alcohol solvent, such as methanol, propanol, butanol, pentanol, hexanol, cyclohexanol, methyl cellosolve, ethyl cellosolve and ethylene glycol, an ether solvent, such as dibutyl ether, tetrahydrofuran, dioxane and anisole, an amide or imide solvent, such as N,N-dimethylformamide, N,N-dimethylacetamide, 1-methyl-2-pyrrolidone and 1-methyl-2-imidazolidinone, a sulfoxide solvent, such as dimethylsulfoxide, and a nitrile solvent, such as acetonitrile) and/or water to prepare a coating liquid, which may be then coated by various coating methods to form the thin film. The solvent may be used solely or as a combination of plural kinds thereof. Among these, a hydrocarbon solvent, a halogenated hydrocarbon solvent and an ether solvent are preferred, toluene, xylene, mesitylene, tetralin, dichlorobenzene and anisole are more preferred, and toluene, xylene, tetralin and anisole are particularly preferred. The concentration of the compound represented by the formula (1) in the coating liquid is preferably from 0.1 to 80% by mass, and more preferably from 0.1 to 10% by mass, by which a film having an arbitrary thickness may be formed.

For forming a film by a solution process, it is necessary to dissolve the materials in the aforementioned solvent, but it is insufficient that the materials are simply dissolved in the solvent. In general, a material to be formed into a film by a vacuum process may be dissolved in a solvent in a certain extent. However, the solution process includes a step of evaporating the solvent to form a thin film, after coating the materials dissolved in a solvent, and most of materials that are not suitable for forming a film by a solution process have high crystallinity, and thus may be disadvantageously crystallized (agglomerated) in the step to fail to provide a favorable thin film. The compound represented by the formula (1) is advantageous also in such a point that the compound may not cause the disadvantageous crystallization (agglomeration).

As the coating solution for a non-light emitting organic semiconductor device of the invention, such an embodiment is also preferred that contains the compound represented by the formula (1), i.e., the compound of the invention, and contains no polymer binder.

The coating solution for a non-light emitting organic semiconductor device of the invention may contain the compound represented by the formula (1), i.e., the compound of the invention, and a polymer binder. In this case, the thin film may be formed in such a manner that the material for forming the layer and the polymer binder are dissolved or dispersed in the suitable solvent described above to prepare a coating liquid, which is then coated by various coating method to form the thin film. The polymer binder may be selected from those described above.

### EXAMPLE

The features of the invention will be described more specifically with reference to examples and comparative examples below. The materials, the amounts used, the ratios, the contents of processes, the procedures of processes, and the like shown in the examples may be appropriately changed unless they deviate the substance of the invention. Accordingly, the scope of the invention is not construed as being limited to the following examples.

### Example 1

### Synthesis Example 1

### Synthesis of Compound 3

The compound 3 as the compound represented by the formula (1) was synthesized by the specific synthesis procedures shown by the following scheme.

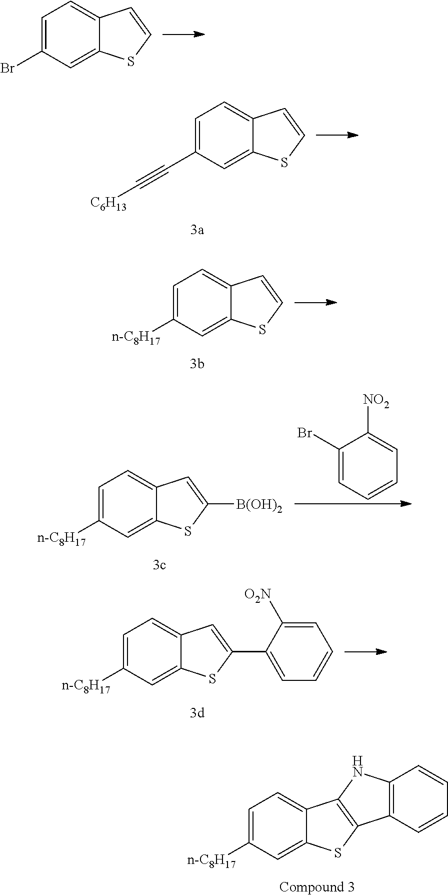

### Synthesis of Compound 3a

A piperidine solution (60 mL) of 6-Bromobenzo[b]thiophene (produced by Sigma-Aldrich Corporation) (3 g), 1-octine (3.1 g), PdCl₂(PPh3)₂ (1 g) and copper(I) iodide (0.53 g) were stirred in a nitrogen atmosphere at 55° C. for 4 hours. The reaction liquid was poured into a mixture of ethyl acetate and water (1/1), and the mixture was filtered with Celite. The aqueous layer of the resulting filtrate was made acidic with a 1N hydrochloric acid aqueous solution, and the filtrate was separated into an organic layer and the aqueous layer. The organic layer was washed with a sodium chloride aqueous solution, dried over magnesium sulfate, and then concentrated under reduced pressure. The residue after the concentration was purified by silica gel column chromatography to provide a compound 3a (3.2 g).

### Synthesis of Compound 3b

The compound 3a (3.0 g), 10% by mass Pd/C (2.6 g) and isopropyl alcohol (35 mL) were placed in an autoclave and stirred under a hydrogen pressure of 5 atm at 50° C. for 3 hours. The reaction liquid was filtered with Celite, and the resulting filtrate was concentrated under reduced pressure. The residue after the concentration was purified by silica gel column chromatography to provide a compound 3b (2.8 g).

### Synthesis of Compound 3c

A 1.6N n-BuLi n-hexane solution (6.1 mL) was added dropwise to a THF solution (100 mL) of the compound 3b (2.0 g) at −78° C. After stirring for 1 hour, trimethoxyborane (1.7 g) was added dropwise thereto, and the mixture was stirred at room temperature for 1 hour. The reaction liquid was poured into a mixture of ethyl acetate and a 1N hydrochloric acid aqueous solution (1/1), and the organic layer was washed with a sodium chloride aqueous solution, dried over magnesium sulfate, and then concentrated under reduced pressure. The residue after the concentration was purified by silica gel column chromatography to provide a compound 3c (1.7 g).

### Synthesis of Compound 3d

1,2-Dimethoxyethane (12 mL) and water (3 mL) were added to the compound 3c (1 g), 2-bromonitrobenzene (0.6 g), 2-dichlorohexylphosphino-2′,6′-dimethoxybiphenyl (SPhos) (0.15 g) and sodium carbonate (0.95 g), to which palladium acetate (20 mg) was added, and then the mixture was stirred in a nitrogen atmosphere under heating to reflux for 2 hours. The reaction liquid was filtered with Celite, to which water was added, and then the mixture was separated into an organic layer and an aqueous layer. The organic layer was washed with a sodium chloride aqueous solution, dried over magnesium sulfate, and concentrated under reduced pressure. The residue after the concentration was purified by silica gel column chromatography to provide a compound 3d (0.86 g).

### Synthesis of Compound 3

The compound 3d (0.8 g) and triphenylphosphine (6.1 g) were stirred under a nitrogen atmosphere at 180° C. for 4 hours. The reaction mixture was purified by silica gel column chromatography to provide a compound 3 (0.54 g). The compound was identified by elemental analysis, NMR and mass spectrum. The result of the identification of the structure of the compound 3 by ¹H-NMR is shown below.

¹H-NMR (DMSO-d₆): 12.05 (1H), 8.00 (1H), 7.81 (1H), 7.76 (1H), 7.56 (1H), 7.33 (1H), 7.27 (1H), 7.15 (1H), 2.74 (2H), 1.66 (2H), 1.31-1.24 (10H), 0.86 (3H)

The other compounds represented by the formula (1) were synthesized in the similar manner as for the compound 3. The result of the identification of the structure of the compound 1 by ¹H-NMR is shown below.

¹H-NMR (DMSO-d₆): 12.04 (1H), 8.01 (1H), 7.80 (1H), 7.76 (1H), 7.55 (1H), 7.35 (1H), 7.30 (1H), 7.15 (1H), 2.71 (2H), 1.65 (2H), 1.31-1.23 (4H), 0.85 (3H)

The result of the identification of the structure of the compound 9 by ¹H-NMR is shown below.

¹H-NMR (CDCl₃): 11.86 (1H), 7.91 (1H), 7.80 (1H), 7.61 (1H), 7.33 (1H), 7.30 (1H), 6.97 (1H), 2.71 (4H), 1.64 (4H), 1.31-1.24 (20H), 0.85 (6H)

The result of the identification of the structure of the compound 81 by ¹H-NMR is shown below.

¹H-NMR (DMSO-d₆): 12.04 (1H), 8.00 (1H), 7.82 (1H), 7.74 (¹H), 7.57 (1H), 7.33 (1H), 7.27 (1H), 7.13 (1H), 2.72 (2H), 1.66 (2H), 1.35-1.20 (16H), 0.86 (3H)

Comparative compounds 1 to 9 used in a semiconductor active layer (organic semiconductor layer) of comparative devices were synthesized according to the methods described in the literatures. The structures of the comparative compounds 1 to 9 are shown below.

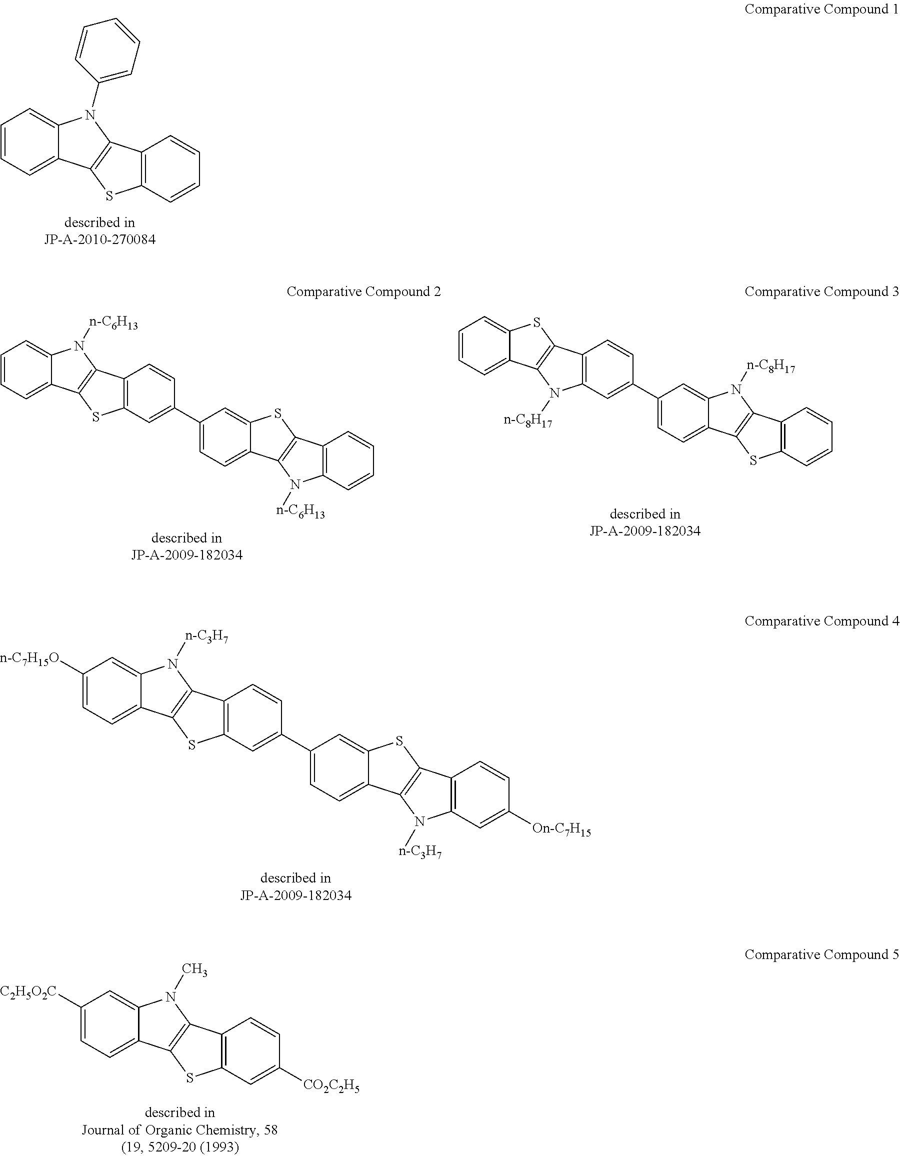

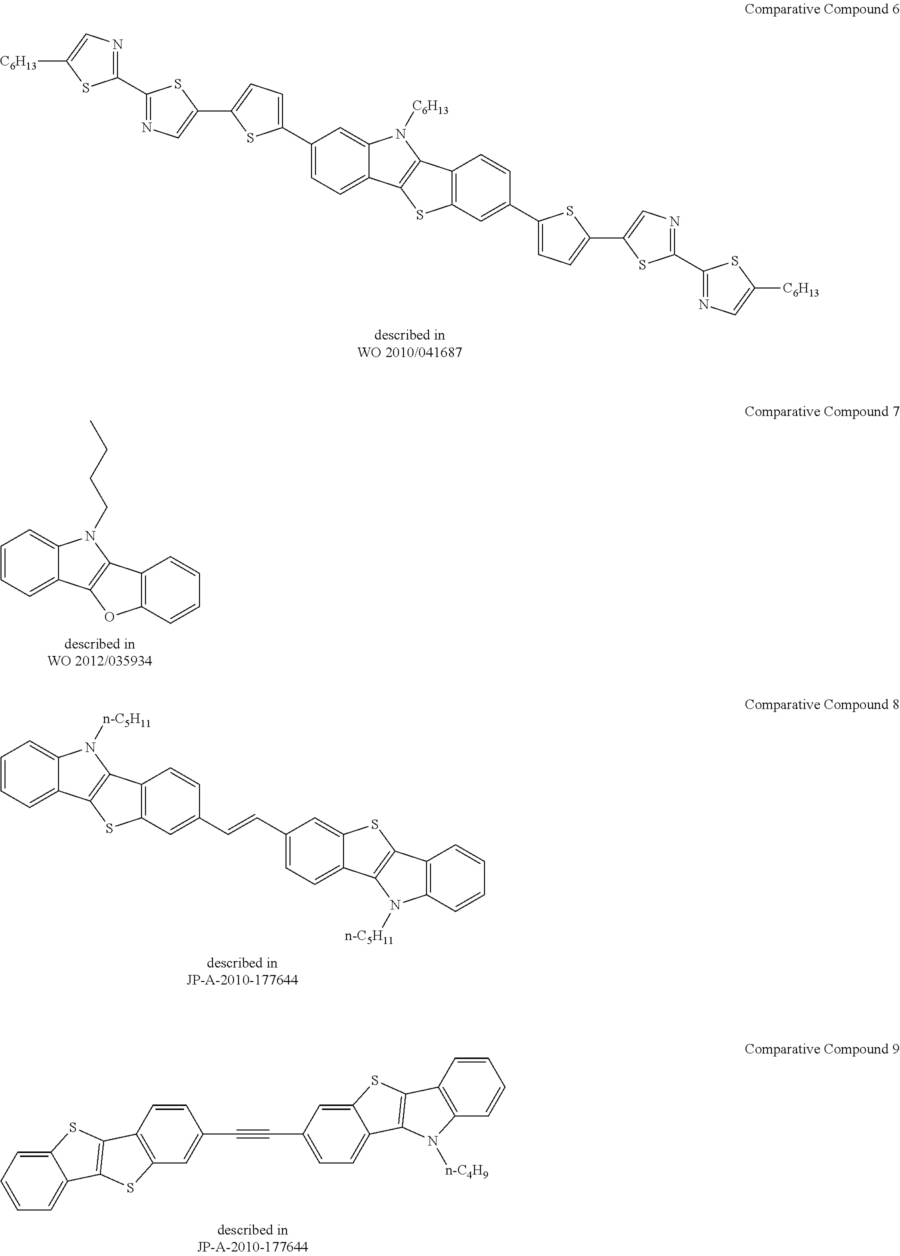

### Production and Evaluation of Devices

All the materials used for producing devices were purified by sublimation, and were confirmed to have a purity (absorption intensity area ratio at 254 nm) of 99.95% or more by high-performance liquid chromatography (TSKgel ODS-100Z, available from Tosoh Corporation).

### Example 2

### Formation of Semiconductor Active Layer (Organic Semiconductor Layer) Only with Compound

The compound of the invention or the comparative compound (1 mg each) and toluene (1 mL) were mixed and heated to 100° C. to prepare a coating solution for a non-light emitting organic semiconductor device. The coating solution was cast on a substrate for measuring FET characteristics heated to 90° C. under nitrogen atmosphere to form an organic semiconductor thin film for a non-light emitting organic semiconductor device, thereby providing an organic thin film transistor device of Example 2 for measuring FET characteristics. The substrate for measuring FET characteristics used was a silicon substrate having a bottom-gate bottom-contact structure having chromium/gold electrodes (gate width W=100 mm, gate length L=100 μm) disposed in an interdigitated form as source and drain electrodes, and SiO₂ (thickness: 200 nm) as an insulating film (the schematic structural illustration shown in FIG. 2).

The FET characteristics of the organic thin film transistor device of Example 2 were evaluated in terms of the carrier mobility and the change in the threshold voltage after repeated driving by using a semiconductor parameter analyzer (4156C, produced by Agilent Technologies, Inc.) having a semi-automatic prober (AX-2000, produced by Vector Semiconductor Co., Ltd.) connected thereto under a normal pressure nitrogen atmosphere.

The results obtained are shown in Table 1 below.

### (a) Carrier Mobility

While applying a voltage of −80 V between the source electrode and the drain electrode of the organic thin film transistor device (FET device), the gate voltage was changed within a range of from 20 to −100 V, and the carrier mobility μ was calculated by the following expression showing the drain current I_{d}.

I_{d}=(W/2L)μCᵢ(V_{g}−Vₜₕ)²

wherein L represents the gate length, W represents the gate width, Cᵢ represents the capacity of the insulating layer per unit area, V_{g} represents the gate voltage, and Vₜₕ represents the threshold voltage. A device that exhibited a carrier mobility of less than 1×10⁻⁵ cm²/Vs was not subjected to the subsequent evaluation of (b) the change in the threshold voltage after repeated driving due to the too poor property thereof.
(b) Change in Threshold Voltage after Repeated Driving

While applying a voltage of −80 V between the source electrode and the drain electrode of the organic thin film transistor device (FET device), the gate voltage was changed 100 times within a range of from 20 to −100 V, and the same measurement as in the measurement (a) above to evaluate the difference (|V₁−V₀|) between the threshold voltage V₀ before repeated driving and the threshold voltage V₁ after repeated driving according to the following three grades. A smaller value thereof shows higher repeated driving stability of the device and thus is preferred. The change in the threshold voltage after repeated driving is preferably the grade A for practical use.

|V₁−V₀|≦5 V A

5 V<|V₁−V₀|≦10 V B

|V₁−V₀|>10 V C

### (c) Molecular Length of Substituent Z

The molecular length of the substituent Z means the length of from the N atom in the N—Z bond in the benzothienoindole structure or the benzofuranoindole structure to the end of the substituent represented by Z. The structure optimization calculation may be performed by the density functional approach (Gaussian 03 (Gaussian, Inc., U.S.), base function: 6-31G*, exchange correlation function: B3LYP/LANL2DZ).

### (d) Crystal Structure

The organic semiconductor compounds used for producing the devices were separately subjected to single crystal growth by a good solvent-poor solvent method, and the presence of a herringbone structure was determined by X-ray crystallography using APEX2, produced by Bruker Corporation. The symbol A shows a herringbone structure, and the symbol B shows other structures.

TABLE 1

| | | | | | Change in | |
|---|---|---|---|---|---|---|
| | | Molecular | Crystal | | threshold | |
| | Organic | length of | structure | Carrier | voltage after | |
| | semiconductor | substituent Z | (herringbone | mobility | repeated | |
| Device No. | material | (Å) | structure) | (cm²/Vs) | driving | Note |
| Device 1 | Compound 1 | 1.0 | A | 8 × 10⁻² | A | invention |
| Device 2 | Compound 4 | 1.0 | A | 3 × 10⁻² | A | invention |
| Device 3 | Compound 5 | 1.0 | A | 5 × 10⁻² | A | invention |
| Device 4 | Compound 8 | 1.0 | A | 6 × 10⁻² | A | invention |
| Device 5 | Compound 9 | 1.0 | A | 4 × 10⁻² | A | invention |
| Device 6 | Compound 13 | 1.0 | A | 7 × 10⁻² | A | invention |
| Device 7 | Compound 16 | 1.0 | A | 5 × 10⁻² | A | invention |
| Device 8 | Compound 19 | 2.1 | A | 1 × 10⁻² | A | invention |
| Device 9 | Compound 20 | 2.1 | A | 2 × 10⁻² | A | invention |
| Device 10 | Compound 21 | 1.0 | A | 8 × 10⁻² | A | invention |
| Device 11 | Compound 22 | 1.0 | A | 7 × 10⁻² | A | invention |
| Device 12 | Compound 25 | 1.0 | A | 3 × 10⁻¹ | A | invention |
| Device 13 | Compound 26 | 1.0 | A | 2 × 10⁻¹ | A | invention |
| Device 14 | Compound 27 | 1.0 | A | 2 × 10⁻¹ | A | invention |
| Device 15 | Compound 28 | 1.0 | A | 1 × 10⁻¹ | A | invention |
| Device 16 | Compound 37 | 1.0 | A | 6 × 10⁻² | A | invention |
| Device 17 | Compound 41 | 1.0 | A | 2 × 10⁻² | A | invention |
| Device 18 | Compound 44 | 1.0 | A | 7 × 10⁻² | A | invention |
| Device 19 | Compound 47 | 1.0 | A | 6 × 10⁻² | A | invention |
| Device 20 | Compound 58 | 2.1 | A | 2 × 10⁻² | A | invention |
| Device 21 | Compound 59 | 1.0 | A | 4 × 10⁻¹ | A | invention |
| Device 22 | Compound 61 | 1.0 | A | 6 × 10⁻¹ | A | invention |
| Device 23 | Compound 66 | 1.0 | A | 1 × 10⁻² | A | invention |
| Device 24 | Compound 76 | 1.0 | A | 8 × 10⁻² | A | invention |
| Comparative | Comparative | 5.3 | B | <1 × 10⁻⁵ | — | comparison |
| Device 1 | Compound 1 | | | | | |
| Comparative | Comparative | 8.4 | B | 2 × 10⁻³ | B | comparison |
| Device 2 | Compound 2 | | | | | |
| Comparative | Comparative | 10.9 | B | 3 × 10⁻³ | B | comparison |
| Device 3 | Compound 3 | | | | | |
| Comparative | Comparative | 4.7 | B | 4 × 10⁻⁴ | B | comparison |
| Device 4 | Compound 4 | | | | | |
| Comparative | Comparative | 2.1 | B | 6 × 10⁻⁴ | C | comparison |
| Device 5 | Compound 5 | | | | | |
| Comparative | Comparative | 8.4 | B | 4 × 10⁻⁵ | B | comparison |
| Device 6 | Compound 6 | | | | | |
| Comparative | Comparative | 6.0 | B | <1 × 10⁻⁵ | — | comparison |
| Device 7 | Compound 7 | | | | | |
| Comparative | Comparative | 7.2 | B | 3 × 10⁻³ | C | comparison |
| Device 8 | Compound 8 | | | | | |
| Comparative | Comparative | 6.0 | B | 4 × 10⁻³ | C | comparison |
| Device 9 | Compound 9 | | | | | |

It was understood from Table 1 that the compound of the invention formed a herringbone structure, and the organic thin film transistor devices using the compounds had a high carrier mobility and a small change in the threshold voltage after repeated driving. Accordingly, it was understood that the compound of the invention was favorably used as an organic semiconductor material for a non-light emitting organic semiconductor device.

On the other hand, the comparative compounds 1 to 9 did not form a herringbone structure, and the organic thin film transistor devices using the compounds had a low carrier mobility and a large change in the threshold voltage after repeated driving.

### Example 3

### Formation of Semiconductor Active Layer (Organic Semiconductor Layer) with Both Compound and Binder

Organic thin film transistor devices for measuring FET characteristics were produced in the same manner as in Example 2 except for using a coating solution prepared in such a manner that the compound of the invention or the comparative compound (1 mg each), 1 mg of PaMS (poly(α-methylstyrene), Mw: 300,000, produced by Sigma-Aldrich, Inc.) and toluene (1 mL) were mixed and heated to 100° C., and then evaluated in the same manner as in Example 2.

The results obtained are shown in Table 2 below.

TABLE 2

| | | | Change in | |
|---|---|---|---|---|
| | | | threshold | |
| | Organic | Carrier | voltage after | |
| | semiconductor | mobility | repeated | |
| Device No. | material | (cm²/Vs) | driving | Note |
| Device 25 | Compound 1 | 4 × 10⁻² | A | invention |
| Device 26 | Compound 28 | 7 × 10⁻² | A | invention |
| Device 27 | Compound 47 | 2 × 10⁻² | A | invention |
| Device 28 | Compound 59 | 9 × 10⁻² | A | invention |
| Device 29 | Compound 61 | 2 × 10⁻¹ | A | invention |
| Device 30 | Compound 76 | 3 × 10⁻² | A | invention |
| Comparative | Comparative | <1 × 10⁻⁵ | — | comparison |
| Device 10 | Compound 1 | | | |
| Comparative | Comparative | 4 × 10⁻⁴ | A | comparison |
| Device 11 | Compound 2 | | | |
| Comparative | Comparative | 6 × 10⁻⁴ | A | comparison |
| Device 12 | Compound 3 | | | |
| Comparative | Comparative | 8 × 10⁻⁵ | A | comparison |
| Device 13 | Compound 4 | | | |
| Comparative | Comparative | 1 × 10⁻⁴ | A | comparison |
| Device 14 | Compound 5 | | | |
| Comparative | Comparative | <1 × 10⁻⁵ | — | comparison |
| Device 15 | Compound 6 | | | |
| Comparative | Comparative | <1 × 10⁻⁵ | — | comparison |
| Device 16 | Compound 7 | | | |
| Comparative | Comparative | 6 × 10⁻⁴ | B | comparison |
| Device 17 | Compound 8 | | | |
| Comparative | Comparative | 5 × 10⁻⁴ | B | comparison |
| Device 18 | Compound 9 | | | |

It was understood from Table 2 that the organic thin film transistor devices having a semiconductor active layer formed by using the compounds of the invention along with the binder had a high carrier mobility and a small change in the threshold voltage after repeated driving. Accordingly, it was understood that the compound of the invention was favorably used as an organic semiconductor material for a non-light emitting organic semiconductor device.

On the other hand, the organic thin film transistor devices having a semiconductor active layer formed by using the comparative compounds 1 to 9 along with the binder had a low carrier mobility. The organic thin film transistor devices having a semiconductor active layer formed by using the comparative compounds 8 and 9 along with the binder also had a large change in the threshold voltage after repeated driving.

It was understood from the observation with an optical microscope of the organic thin film transistor devices obtained in Example 3 that the thin films using PαMS as a binder all had considerably high smoothness and uniformity of the film.

It was understood from these results that the comparative devices having a semiconductor active layer formed with the composite system of the binder and the comparative compound had a considerably low carrier mobility, whereas the organic thin film transistor devices of the invention having a semiconductor active layer formed with both the compound of the invention and the binder had a good carrier mobility, a small change in the threshold voltage after repeated driving, and considerably high smoothness and uniformity of the film.

### Example 4

### Formation of Semiconductor Active Layer (Organic Semiconductor Layer)

A silicon wafer having a gate insulating film of SiO₂ (thickness: 370 nm) was subjected to a surface treatment with octyltrichlorosilane.

The compound of the invention or the comparative compound (1 mg each) and toluene (1 mL) were mixed and heated to 100° C. to prepare a coating solution for a non-light emitting organic semiconductor device. The coating solution was cast on the octyltrichlorosilane-treated silicon wafer heated to 90° C. to form an organic semiconductor thin film for a non-light emitting organic semiconductor device.

On the surface of the thin film thus formed, gold was vapor-deposited through a mask to form source and drain electrodes, thereby providing an organic thin film transistor device having a bottom-gate top-contact structure having a gate width W of 5 mm and a gate length L of 80 μm (the schematic structural illustration shown in FIG. 1).

The FET characteristics of the organic thin film transistor device of Example 4 were evaluated in terms of the carrier mobility and the change in the threshold voltage after repeated driving by using a semiconductor parameter analyzer (4156C, produced by Agilent Technologies, Inc.) having a semi-automatic prober (AX-2000, produced by Vector Semiconductor Co., Ltd.) connected thereto under a normal pressure nitrogen atmosphere.

The results obtained are shown in Table 3 below.

TABLE 3

| | | | Change in | |
|---|---|---|---|---|
| | | | threshold | |
| | Organic | Carrier | voltage after | |
| | semiconductor | mobility | repeated | |
| Device No. | material | (cm²/Vs) | driving | Note |
| Device 30 | Compound 1 | 6 × 10⁻¹ | A | invention |
| Device 31 | Compound 3 | 9 × 10⁻² | A | invention |
| Device 32 | Compound 6 | 2 × 10⁻¹ | A | invention |
| Device 33 | Compound 10 | 1 × 10⁻¹ | A | invention |
| Device 34 | Compound 14 | 8 × 10⁻¹ | A | invention |
| Device 35 | Compound 17 | 2 × 10⁻¹ | A | invention |
| Device 36 | Compound 25 | 1.0 | A | invention |
| Device 37 | Compound 27 | 1.1 | A | invention |
| Device 38 | Compound 43 | 4 × 10⁻¹ | A | invention |
| Device 39 | Compound 48 | 3 × 10⁻¹ | A | invention |
| Device 40 | Compound 61 | 1.4 | A | invention |
| Device 41 | Compound 75 | 8 × 10⁻¹ | A | invention |
| Comparative | Comparative | <1 × 10⁻⁵ | — | comparison |
| Device 19 | Compound 1 | | | |
| Comparative | Comparative | 8 × 10⁻³ | B | comparison |
| Device 20 | Compound 2 | | | |
| Comparative | Comparative | 9 × 10⁻³ | B | comparison |
| Device 21 | Compound 3 | | | |
| Comparative | Comparative | 1 × 10⁻³ | B | comparison |
| Device 22 | Compound 4 | | | |
| Comparative | Comparative | 3 × 10⁻³ | C | comparison |
| Device 23 | Compound 5 | | | |
| Comparative | Comparative | 2 × 10⁻⁴ | B | comparison |
| Device 24 | Compound 6 | | | |
| Comparative | Comparative | <1 × 10⁻⁵ | — | comparison |
| Device 25 | Compound 7 | | | |
| Comparative | Comparative | 9 × 10⁻³ | C | comparison |
| Device 26 | Compound 8 | | | |
| Comparative | Comparative | 2 × 10⁻² | C | comparison |
| Device 27 | Compound 9 | | | |

It was understood from Table 3 that the organic thin film transistor devices using the compounds of the invention had a high carrier mobility and a small change in the threshold voltage after repeated driving. Accordingly, it was understood that the compound of the invention was favorably used as an organic semiconductor material for a non-light emitting organic semiconductor device.

On the other hand, the organic thin film transistor devices using the comparative compounds 1 and 4 to 7 had a low carrier mobility. The organic thin film transistor devices using the comparative compounds 2 to 6, 8 and 9 had a large change in the threshold voltage after repeated driving.

In general, a top-contact device (Example 4) has a tendency that the carrier mobility is nearly one digit higher than a bottom-contact device (Examples 2 and 3) since a top-contact device has good contact with the electrode. The comparative devices 20 and 21 each had a relatively high mobility, but it was found that these results were due to the top-contact device, and there was a nearly one digit difference in the mobility in the comparison to the devices using the compounds of the invention that had the same top-contact structure.

While the present invention has been described in detail and with reference to specific embodiments thereof, it will be apparent to one skilled in the art that various changes and modifications can be made therein without departing from the spirit and scope thereof.

The present disclosure relates to the subject matter contained in International Application No. PCT/JP2014/052868 filed on Feb. 7, 2014; Japanese Patent Application No. 2013-022484 filed on Feb. 7, 2013, and Japanese Patent Application No. 2014-020141 filed on Feb. 5, 2014, the contents of which are expressly incorporated herein by reference in their entirety. All the publications referred to in the present specification are also expressly incorporated herein by reference in their entirety.

The foregoing description of preferred embodiments of the invention has been presented for purposes of illustration and description, and is not intended to be exhaustive or to limit the invention to the precise form disclosed. The description was selected to best explain the principles of the invention and their practical application to enable others skilled in the art to best utilize the invention in various embodiments and various modifications as are suited to the particular use contemplated. It is intended that the scope of the invention not be limited by the specification, but be defined claims.

What is claimed is:

1. An organic thin film transistor containing a compound represented by the following formula (1) in a semiconductor active layer: 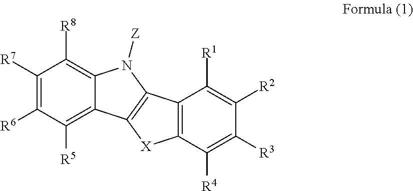
wherein in the formula (1), X represents a S atom or an O atom; Z represents a substituent that has a length of 3.7 Å or less from the N atom to the end of the substituent; and R¹ to R⁸ each independently represent a hydrogen atom or a substituent, provided that at least one of R¹ to R⁸ represents a substituent represented by the following formula (W):
-L-R Formula (W)
wherein in the formula (W), L represents a divalent linking group represented by any one of the following formulae (L-1) to (L-12), or a divalent linking group containing 2 or more divalent linking groups each represented by any one of the following formulae (L-1) to (L-12) bonded to each other; and R represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R has a number of carbon atoms of 2 or more in the case where L represents a divalent linking group represented by any one of the formulae (L-1) to (L-3), or 4 or more in the case where L represents a divalent linking group represented by any one of the formulae (L-4) to (L-12), and R represents a substituted or unsubstituted trialkylsilyl group only in the case where L adjacent to R represents a divalent linking group represented by the formula (L-3): 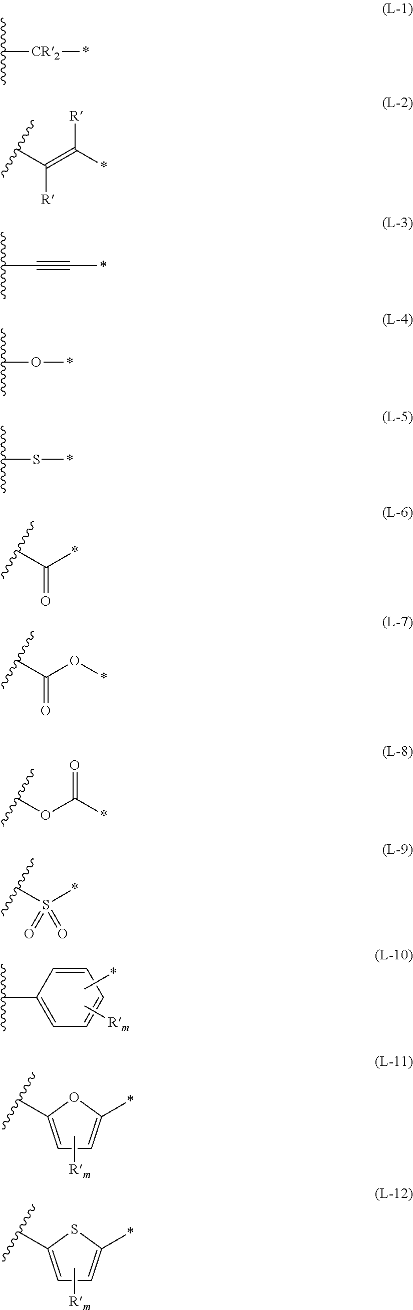
wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

2. The organic thin film transistor according to claim 1, wherein at least one of R², R³, R⁶ and R⁷ represents a substituent represented by the formula (W).

3. The organic thin film transistor according to claim 1, wherein the compound represented by the formula (1) is a compound represented by the following formula (2-1) or (2-2) 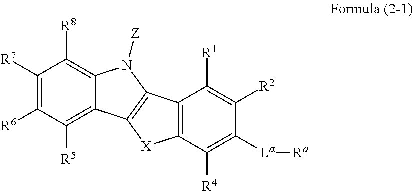
wherein in the formula (2-1), X represents a S atom or an O atom; Z represents a substituent that has a length of 3.7 Å or less from the N atom to the end of the substituent; R¹, R² and R⁴ to R⁸ each independently represent a hydrogen atom or a substituent, provided that R⁷ is not a substituent represented by -L^{a}-R^{a}; L^{a} represents a divalent linking group represented by any one of the following formulae (L-1) to (L-12), or a divalent linking group containing 2 or more divalent linking groups each represented by any one of the following formulae (L-1) to (L-12) bonded to each other; and R^{a} represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R^{a} has a number of carbon atoms of 2 or more in the case where L^{a} represents a divalent linking group represented by any one of the formulae (L-1) to (L-3), or 4 or more in the case where L^{a} represents a divalent linking group represented by any one of the formulae (L-4) to (L-12), and R^{a} represents a substituted or unsubstituted trialkylsilyl group only in the case where L^{a} adjacent to R^{a} represents a divalent linking group represented by the formula (L-3), 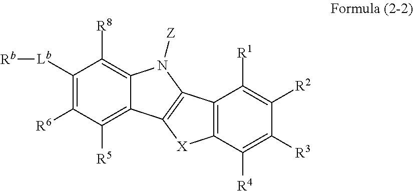
wherein in the formula (2-2), X represents a S atom or an O atom; Z represents a substituent that has a length of 3.7 Å or less from the N atom to the end of the substituent; R¹ to R⁶ and R⁸ each independently represent a hydrogen atom or a substituent, provided that R³ is not a substituent represented by -L^{b}-R^{b}; L^{b} represents a divalent linking group represented by any one of the following formulae (L-1) to (L-12), or a divalent linking group containing 2 or more divalent linking groups each represented by any one of the following formulae (L-1) to (L-12) bonded to each other; and R^{b} represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R^{b} has a number of carbon atoms of 2 or more in the case where L^{b} represents a divalent linking group represented by any one of the formulae (L-1) to (L-3), or 4 or more in the case where L^{b} represents a divalent linking group represented by any one of the formulae (L-4) to (L-12), and R^{b} represents a substituted or unsubstituted trialkylsilyl group only in the case where L^{b} adjacent to R^{b} represents a divalent linking group represented by the formula (L-3): 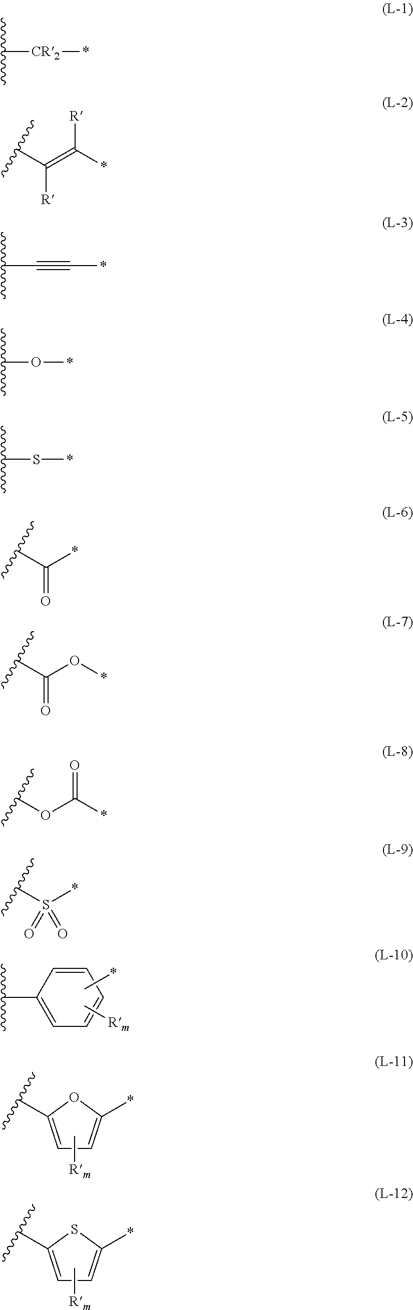
wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

4. The organic thin film transistor according to claim 1, wherein the compound represented by the formula (1) is a compound represented by the following formula (3) 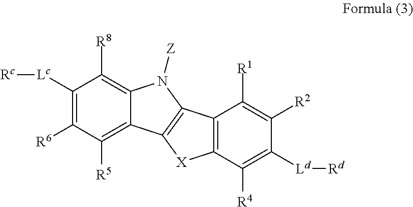
wherein in the formula (3), X represents a S atom or an O atom; Z represents a substituent that has a length of 3.7 Å or less from the N atom to the end of the substituent; R¹, R², R⁴ to R⁶ and R⁸ each independently represent a hydrogen atom or a substituent; L^{c} and L^{d} each independently represent a divalent linking group represented by any one of the following formulae (L-1) to (L-12), or a divalent linking group containing 2 or more divalent linking groups each represented by any one of the following formulae (L-1) to (L-12) bonded to each other; and R^{c} and R^{d} each independently represent a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl groups represented by R^{c} and R^{d} each have a number of carbon atoms of 2 or more in the case where L^{c} and L^{d} respectively represent a divalent linking group represented by any one of the formulae (L-1) to (L-3), or 4 or more in the case where L^{c} and L^{d} respectively represent a divalent linking group represented by any one of the formulae (L-4) to (L-12), and R^{c} and R^{d} each represent a substituted or unsubstituted trialkylsilyl group only in the case where L^{c} and L^{d} adjacent to R^{c} and R^{d} respectively represent a divalent linking group represented by the formula (L-3): 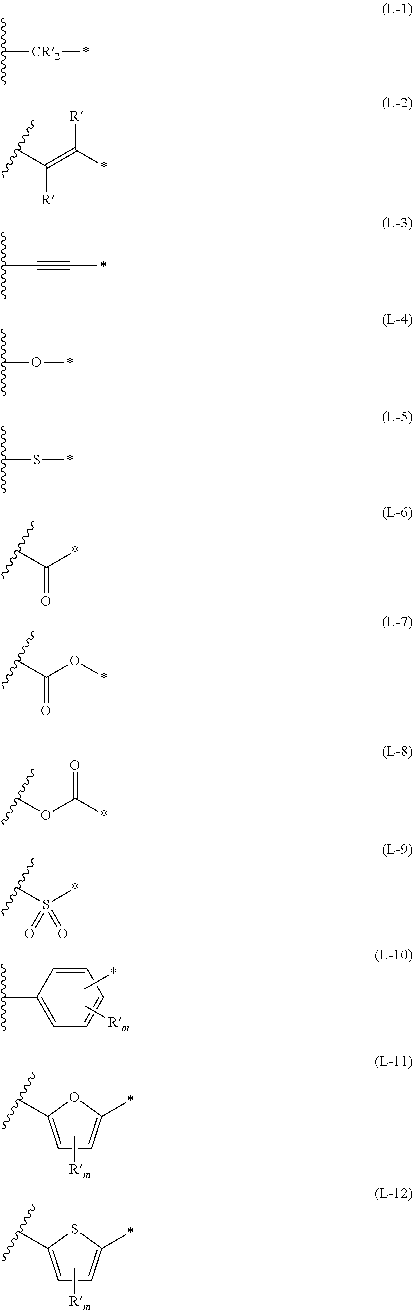
wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

5. The organic thin film transistor according to claim 3, wherein in the formula (2-1), (2-2) or (3), Z represents a hydrogen atom, a substituted or unsubstituted alkyl group having 2 or less carbon atoms, a substituted or unsubstituted alkynyl group having 2 or less carbon atoms, a substituted or unsubstituted alkenyl group having 2 or less carbon atoms, or a substituted or unsubstituted acyl group having 2 or less carbon atoms.

6. The organic thin film transistor according to claim 3, wherein in the formula (2-1), (2-2) or (3), R¹, R⁴, R⁵ and R⁸ each independently represent a hydrogen atom, a fluorine atom, a substituted or unsubstituted alkyl group having from 1 to 3 carbon atoms, a substituted or unsubstituted alkynyl group having from 2 to 3 carbon atoms, a substituted or unsubstituted alkenyl group having from 2 to 3 carbon atoms, a substituted or unsubstituted alkoxy group having from 1 to 2 carbon atoms, or a substituted or unsubstituted methylthio group.

7. The organic thin film transistor according to claim 3, wherein in the formula (2-1), (2-2) or (3), all of L^{a}, L^{b}, L^{c} and L^{d} each represent a divalent linking group represented by any one of the formulae (L-1) to (L-3), (L-10), (L-11) or (L-12), or a divalent linking group containing 2 or more of the divalent linking groups bonded to each other.

8. The organic thin film transistor according to claim 3, wherein in the formula (2-1), (2-2) or (3), all of L^{a}, L^{b}, L^{c} and L^{d} each independently represent a divalent linking group represented by the formula (L-1) or (L-10).

9. The organic thin film transistor according to claim 3, wherein in the formula (2-1), (2-2) or (3), all R^{a}, R^{b}, and R^{d} each independently represent a substituted or unsubstituted alkyl group.

10. The organic thin film transistor according to claim 3, wherein in the formula (2-1) or (2-2), R^{a} and R^{b} each independently represent a branched alkyl group; or
L^{a} and L^{b} each independently represent a divalent linking group represented by the formula (L-1), and at least one of R′ in the divalent linking groups represented by the formula (L-1) represents an alkyl group.

11. The organic thin film transistor according to claim 4, wherein in the formula (3), R^{c} and R^{d} each independently represent a linear alkyl group, provided that in the case where L^{c} and L^{d} each independently represent a divalent linking group represented by the formula (L-1), all R′ in the divalent linking groups represented by the formula (L-1) each represent a hydrogen atom.

12. A compound represented by the following formula (1): 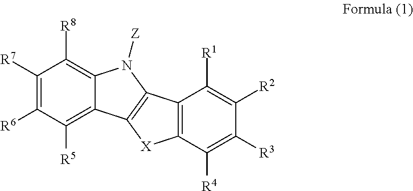
wherein in the formula (1), X represents a S atom or an O atom; Z represents a substituent that has a length of 3.7 Å or less from the N atom to the end of the substituent; and R¹ to R⁸ each independently represent a hydrogen atom or a substituent, provided that at least one of R¹ to R⁸ represents a substituent represented by the following formula (W):
-L-R Formula (W)
wherein in the formula (W), L represents a divalent linking group represented by any one of the following formulae (L-1) to (L-12), or a divalent linking group containing 2 or more divalent linking groups each represented by any one of the following formulae (L-1) to (L-12) bonded to each other; and R represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R has a number of carbon atoms of 2 or more in the case where L represents a divalent linking group represented by any one of the formulae (L-1) to (L-3), or 4 or more in the case where L represents a divalent linking group represented by any one of the formulae (L-4) to (L-12), and R represents a substituted or unsubstituted trialkylsilyl group only in the case where L adjacent to R represents a divalent linking group represented by the formula (L-3): 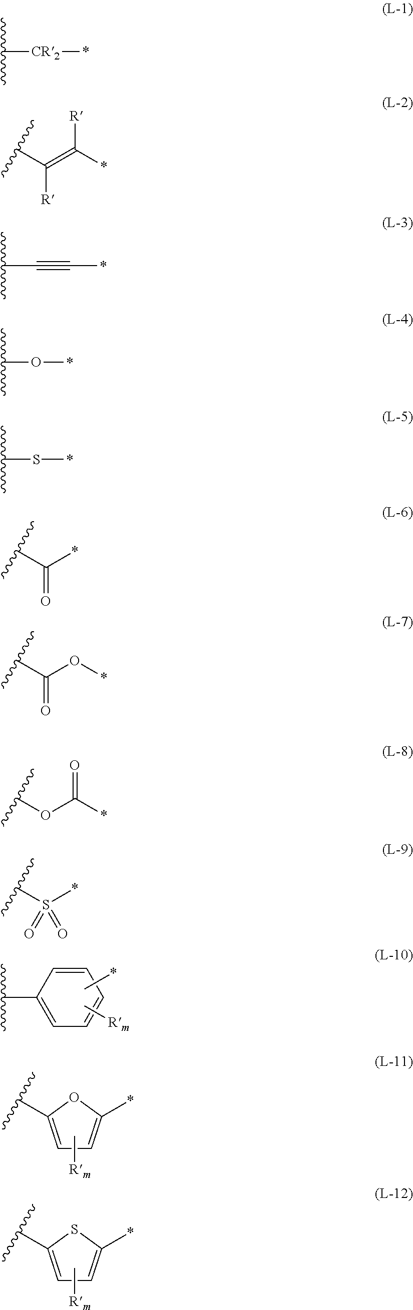
wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

13. The compound according to claim 12, wherein at least one of R², R³, R⁶ and R⁷ represents a substituent represented by the formula (W).

14. The compound according to claim 1, wherein the compound represented by the formula (1) is a compound represented by the following formula (2-1) or (2-2): 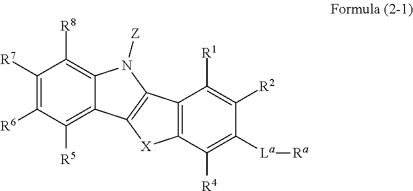
wherein in the formula (2-1), X represents a S atom or an O atom; Z represents a substituent that has a length of 3.7 Å or less from the N atom to the end of the substituent; R¹, R² and R⁴ to R⁸ each independently represent a hydrogen atom or a substituent, provided that R⁷ is not a substituent represented by -L^{a}-R^{a}; L^{a} represents a divalent linking group represented by any one of the following formulae (L-1) to (L-12), or a divalent linking group containing 2 or more divalent linking groups each represented by any one of the following formulae (L-1) to (L-12) bonded to each other; and R^{a} represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R^{a} has a number of carbon atoms of 2 or more in the case where L^{a} represents a divalent linking group represented by any one of the formulae (L-1) to (L-3), or 4 or more in the case where L^{a} represents a divalent linking group represented by any one of the formulae (L-4) to (L-12), and R^{a} represents a substituted or unsubstituted trialkylsilyl group only in the case where L^{a} adjacent to R^{a} represents a divalent linking group represented by the formula (L-3), 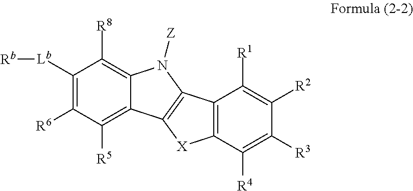
wherein in the formula (2-2), X represents a S atom or an O atom; Z represents a substituent that has a length of 3.7 Å or less from the N atom to the end of the substituent; R¹ to R⁶ and R⁸ each independently represent a hydrogen atom or a substituent, provided that R³ is not a substituent represented by -L^{b}-R^{b}; L^{b} represents a divalent linking group represented by any one of the following formulae (L-1) to (L-12), or a divalent linking group containing 2 or more divalent linking groups each represented by any one of the following formulae (L-1) to (L-12) bonded to each other; and R^{b} represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R^{b} has a number of carbon atoms of 2 or more in the case where L^{b} represents a divalent linking group represented by any one of the formulae (L-1) to (L-3), or 4 or more in the case where L^{b} represents a divalent linking group represented by any one of the formulae (L-4) to (L-12), and R^{b} represents a substituted or unsubstituted trialkylsilyl group only in the case where L^{b} adjacent to R^{b} represents a divalent linking group represented by the formula (L-3): 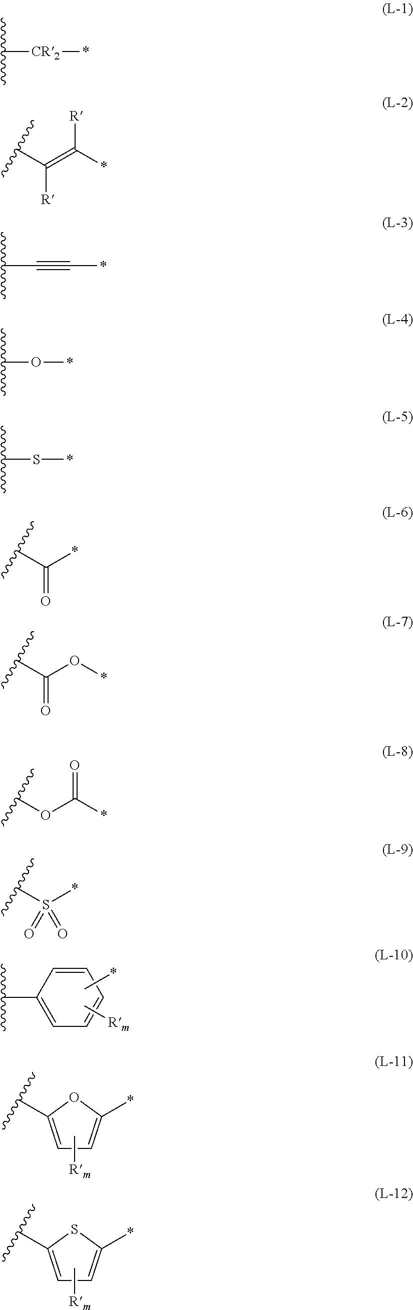
wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

15. The compound according to claim 12, wherein the compound represented by the formula (1) is a compound represented by the following formula (3): 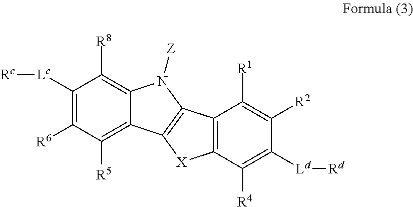
wherein in the formula (3), X represents a S atom or an O atom; Z represents a substituent that has a length of 3.7 Å or less from the N atom to the end of the substituent; R¹, R², R⁴ to R⁶ and R⁸ each independently represent a hydrogen atom or a substituent; L° and L^{d} each independently represent a divalent linking group represented by any one of the following formulae (L-1) to (L-12), or a divalent linking group containing 2 or more divalent linking groups each represented by any one of the following formulae (L-1) to (L-12) bonded to each other; and R^{c} and R^{d} each independently represent a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl groups represented by R^{c} and R^{d} each have a number of carbon atoms of 2 or more in the case where L^{c} and L^{d} respectively represent a divalent linking group represented by any one of the formulae (L-1) to (L-3), or 4 or more in the case where L^{c} and L^{d} respectively represent a divalent linking group represented by any one of the formulae (L-4) to (L-12), and R^{c} and R^{d} each represent a substituted or unsubstituted trialkylsilyl group only in the case where L^{c} and L^{d} adjacent to R^{c} and R^{d} respectively represent a divalent linking group represented by the formula (L-3): 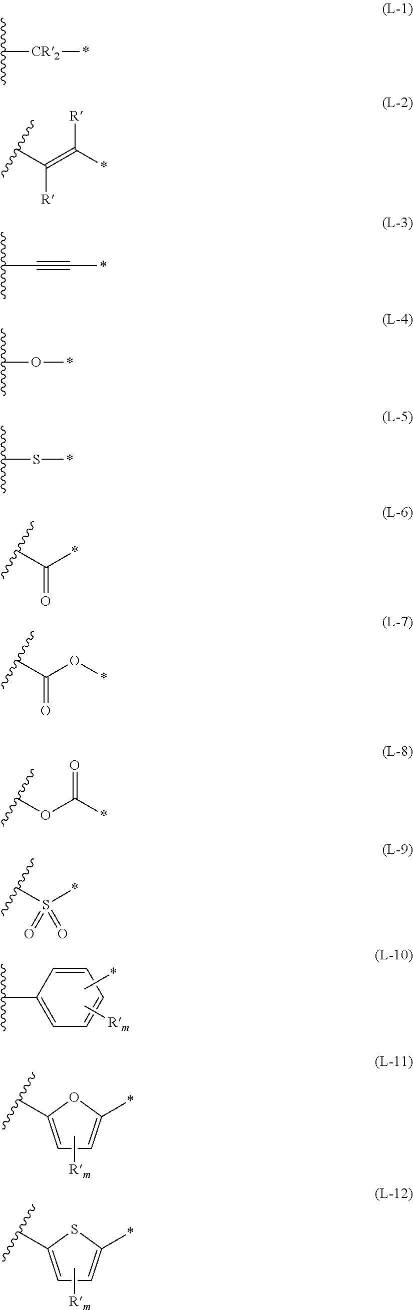
wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

16. The compound according to claim 14, wherein in the formula (2-1), (2-2) or (3), Z represents a hydrogen atom, a substituted or unsubstituted alkyl group having 2 or less carbon atoms, a substituted or unsubstituted alkynyl group having 2 or less carbon atoms, a substituted or unsubstituted alkenyl group having 2 or less carbon atoms, or a substituted or unsubstituted acyl group having 2 or less carbon atoms.

17. The compound according to claim 14, wherein in the formula (2-1), (2-2) or (3), R¹, R⁴, R⁵ and R⁸ each independently represent a hydrogen atom, a fluorine atom, a substituted or unsubstituted alkyl group having from 1 to 3 carbon atoms, a substituted or unsubstituted alkynyl group having from 2 to 3 carbon atoms, a substituted or unsubstituted alkenyl group having from 2 to 3 carbon atoms, a substituted or unsubstituted alkoxy group having from 1 to 2 carbon atoms, or a substituted or unsubstituted methylthio group.

18. The compound according to claim 14, wherein in the formula (2-1), (2-2) or (3), all of L^{a}, L^{b}, L^{a} and L^{d} each represent a divalent linking group represented by any one of the formulae (L-1) to (L-3), (L-10), (L-11) or (L-12), or a divalent linking group containing 2 or more of the divalent linking groups bonded to each other.

19. The compound according to claim 14, wherein in the formula (2-1), (2-2) or (3), all of L^{a}, L^{b}, L^{a} and L^{d} each independently represent a divalent linking group represented by the formula (L-1) or (L-10).

20. The compound according to claim 14, wherein in the formula (2-1), (2-2) or (3), all R^{a}, R^{b}, R^{a} and R^{d} each independently represent a substituted or unsubstituted alkyl group.

21. The compound according to claim 14, wherein in the formula (2-1) or (2-2), R^{a} and R^{b} each independently represent a branched alkyl group; or
L^{a} and L^{b} each independently represent a divalent linking group represented by the formula (L-1), and at least one of R′ in the divalent linking groups represented by the formula (L-1) represents an alkyl group.

22. The compound according to claim 15, wherein in the formula (3), R^{c} and R^{d} each independently represent a linear alkyl group, provided that in the case where L^{c} and L^{d} each independently represent a divalent linking group represented by the formula (L-1), all R′ in the divalent linking groups represented by the formula (L-1) each represent a hydrogen atom.

23. An organic semiconductor material for a non-light emitting organic semiconductor device, containing a compound represented by the following formula (1): 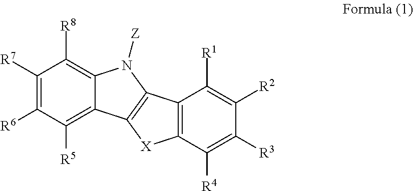
wherein in the formula (1), X represents a S atom or an O atom; Z represents a substituent that has a length of 3.7 Å or less from the N atom to the end of the substituent; and R¹ to R⁸ each independently represent a hydrogen atom or a substituent, provided that at least one of R¹ to R⁸ represents a substituent represented by the following formula (W):
-L-R Formula (W)
wherein in the formula (W), L represents a divalent linking group represented by any one of the following formulae (L-1) to (L-12), or a divalent linking group containing 2 or more divalent linking groups each represented by any one of the following formulae (L-1) to (L-12) bonded to each other; and R represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R has a number of carbon atoms of 2 or more in the case where L represents a divalent linking group represented by any one of the formulae (L-1) to (L-3), or 4 or more in the case where L represents a divalent linking group represented by any one of the formulae (L-4) to (L-12), and R represents a substituted or unsubstituted trialkylsilyl group only in the case where L adjacent to R represents a divalent linking group represented by the formula (L-3): 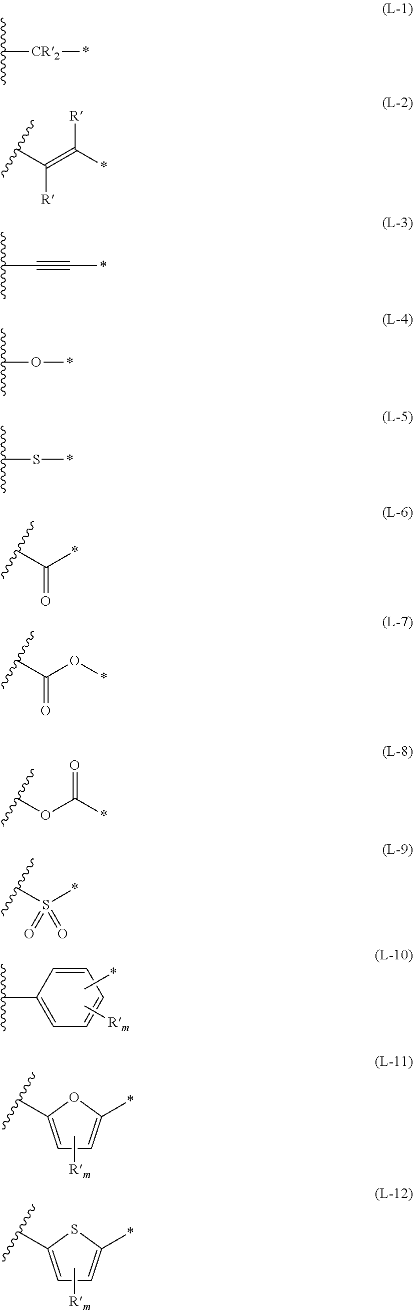
wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

24. A material for an organic thin film transistor, containing a compound represented by the following formula (1): 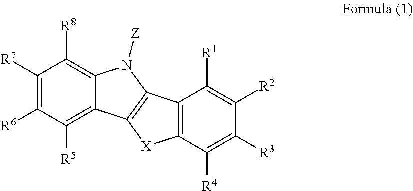
wherein in the formula (1), X represents a S atom or an O atom; Z represents a substituent that has a length of 3.7 Å or less from the N atom to the end of the substituent; and R¹ to R⁸ each independently represent a hydrogen atom or a substituent, provided that at least one of R¹ to R⁸ represents a substituent represented by the following formula (W):
-L-R Formula (W)
wherein in the formula (W), L represents a divalent linking group represented by any one of the following formulae (L-1) to (L-12), or a divalent linking group containing 2 or more divalent linking groups each represented by any one of the following formulae (L-1) to (L-12) bonded to each other; and R represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R has a number of carbon atoms of 2 or more in the case where L represents a divalent linking group represented by any one of the formulae (L-1) to (L-3), or 4 or more in the case where L represents a divalent linking group represented by any one of the formulae (L-4) to (L-12), and R represents a substituted or unsubstituted trialkylsilyl group only in the case where L adjacent to R represents a divalent linking group represented by the formula (L-3): 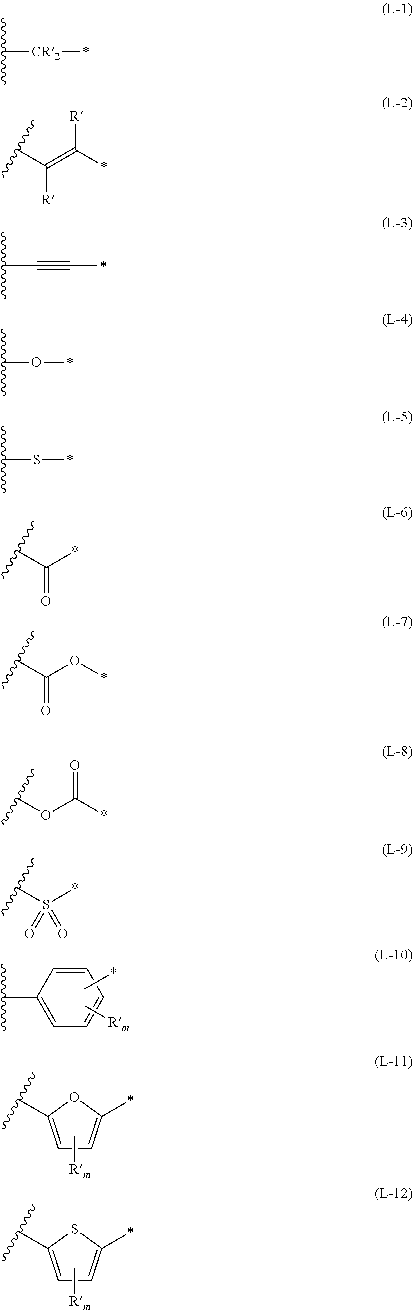
wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

25. A coating solution for a non-light emitting organic semiconductor device, containing a compound represented by the following formula (1): 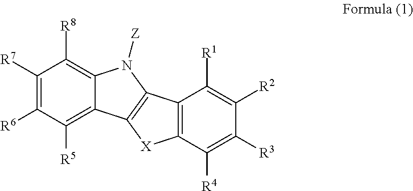
wherein in the formula (1), X represents a S atom or an O atom; Z represents a substituent that has a length of 3.7 Å or less from the N atom to the end of the substituent; and R¹ to R⁸ each independently represent a hydrogen atom or a substituent, provided that at least one of R¹ to R⁸ represents a substituent represented by the following formula (W):
-L-R Formula (W)
wherein in the formula (W), L represents a divalent linking group represented by any one of the following formulae (L-1) to (L-12), or a divalent linking group containing 2 or more divalent linking groups each represented by any one of the following formulae (L-1) to (L-12) bonded to each other; and R represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R has a number of carbon atoms of 2 or more in the case where L represents a divalent linking group represented by any one of the formulae (L-1) to (L-3), or 4 or more in the case where L represents a divalent linking group represented by any one of the formulae (L-4) to (L-12), and R represents a substituted or unsubstituted trialkylsilyl group only in the case where L adjacent to R represents a divalent linking group represented by the formula (L-3): 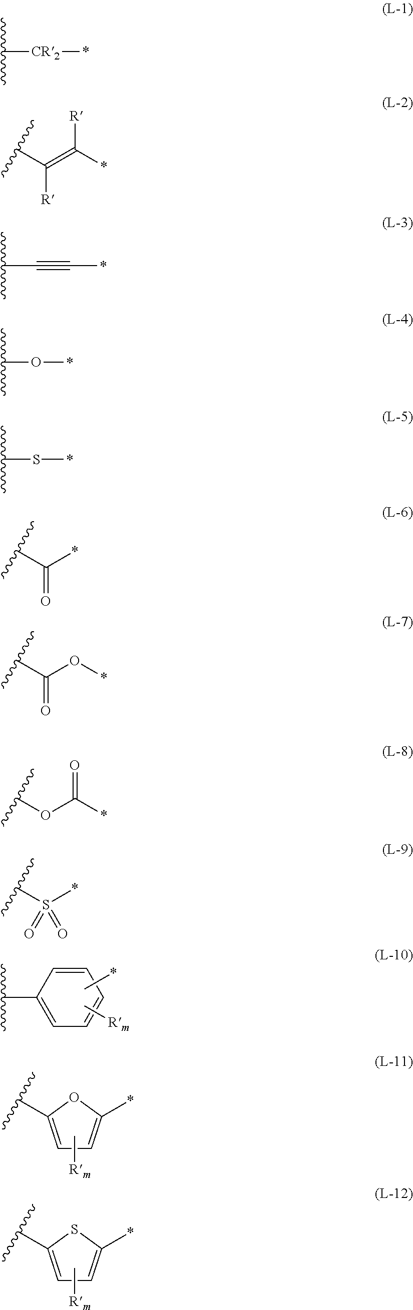
wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

26. The coating solution for a non-light emitting organic semiconductor device according to claim 25, containing the compound represented by the formula (1) and a polymer binder.

27. An organic semiconductor thin film for a non-light emitting organic semiconductor device, containing a compound represented by the following formula (1): 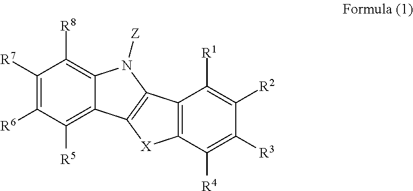
wherein in the formula (1), X represents a S atom or an O atom; Z represents a substituent that has a length of 3.7 Å or less from the N atom to the end of the substituent; and R¹ to R⁸ each independently represent a hydrogen atom or a substituent, provided that at least one of R′ to R⁸ represents a substituent represented by the following formula (W):
-L-R Formula (W)
wherein in the formula (W), L represents a divalent linking group represented by any one of the following formulae (L-1) to (L-12), or a divalent linking group containing 2 or more divalent linking groups each represented by any one of the following formulae (L-1) to (L-12) bonded to each other; and R represents a substituted or unsubstituted alkyl group, an oligooxyethylene group having a repeating number v of an oxyethylene unit of 2 or more, an oligosiloxane group having 2 or more silicon atoms, or a substituted or unsubstituted trialkylsilyl group, provided that the substituted or unsubstituted alkyl group represented by R has a number of carbon atoms of 2 or more in the case where L represents a divalent linking group represented by any one of the formulae (L-1) to (L-3), or 4 or more in the case where L represents a divalent linking group represented by any one of the formulae (L-4) to (L-12), and R represents a substituted or unsubstituted trialkylsilyl group only in the case where L adjacent to R represents a divalent linking group represented by the formula (L-3): 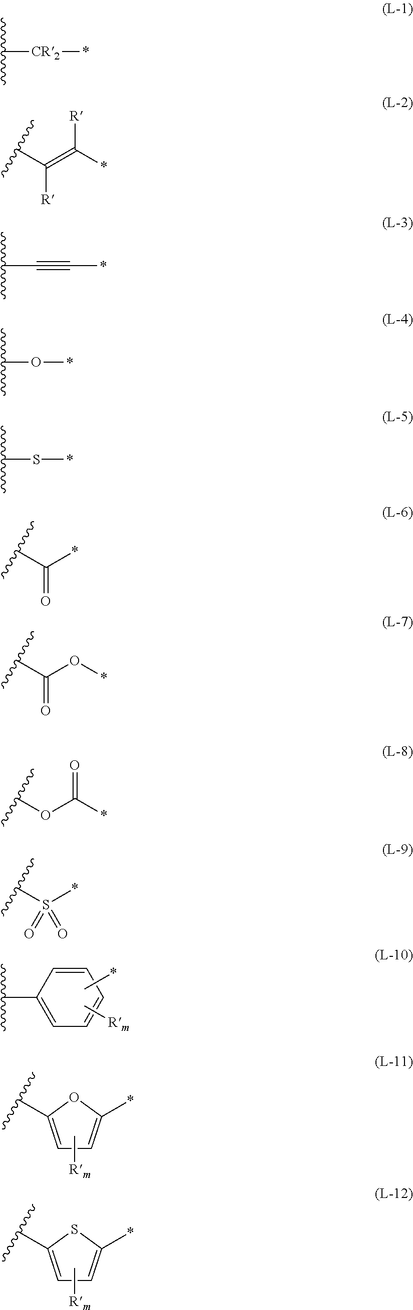
wherein in the formulae (L-1) to (L-12), the wavy line represents a position bonded to the benzothienoindole or benzofuranoindole skeleton; in the formula (L-10), m represents 4; in the formulae (L-11) and (L-12), m represents 2; and in the formulae (L-1), (L-2), (L-10), (L-11) and (L-12), R′ each independently represent a hydrogen atom or a substituent.

28. An organic semiconductor thin film for a non-light emitting organic semiconductor device according to claim 27, containing the compound represented by the formula (1) and a polymer binder.

29. The organic semiconductor thin film for a non-light emitting organic semiconductor device according to claim 27, which is produced by a solution coating method.
